(19) United States
(12) Patent Application Publication
(10) Pub. No.: US 2022/0287838 A1
(43) Pub. Date: Sep. 15, 2022

(54) DELIVERY APPARATUS AND METHODS FOR IMPLANTING PROSTHETIC HEART VALVES

(71) Applicant: Edwards Lifesciences Corporation, Irvine, CA (US)

(72) Inventors: Oren Cohen, Kadima (IL); Tomer Saar, Pardes Hanna-Karkur (IL); Elazar Levi Schwarcz, Netanya (IL); Ofir Witzman, Kfar Saba (IL); Eitan Atias, Tel Aviv (IL); Noam Miller, Givatayim (IL); Eran Goldberg, Nesher (IL); Yair A. Neumann, Moshav Sede Varburg (IL)

(73) Assignee: Edwards Lifesciences Corporation, Irvine, CA (US)

(21) Appl. No.: 17/829,769

(22) Filed: Jun. 1, 2022

Related U.S. Application Data
(63) Continuation of application No. PCT/US2020/063104, filed on Dec. 3, 2020.
(60) Provisional application No. 62/945,039, filed on Dec. 6, 2019.

(51) Int. Cl. A61F 2/24; A61F 2/95

(52) U.S. Cl. CPC A61F 2/2436; A61F 2/2418; A61F 2/9517; A61F 2/243; A61F 2/2466; A61F 2/2439; A61F 2250/001

(57) ABSTRACT

A delivery apparatus for implanting a prosthetic valve includes a handle, a first shaft, a plurality of actuation shafts, and a control mechanism. The first shaft has one or more lumens extending from the first end portion to the second end portion. The actuation shafts each have a proximal end portion and a distal end portion, and the actuation shafts extend through the one or more lumens of the first shaft. The control mechanism is coupled to the actuation shafts and to the handle. The control mechanism is configured such that the actuation shafts can move axially relative to each other in a first operational mode and such that the actuation shafts can be moved axially simultaneously in a second operational mode. Additionally or alternatively, the first shaft can include a plurality of helical lumens configured for receiving the actuation shafts.

### CROSS REFERENCE TO RELATED APPLICATION

This application is a continuation of a PCT Patent Application No. PCT/US2020/063104, entitled “DELIVERY APPARATUS AND METHODS FOR IMPLANTING PROSTHETIC HEART VALVES,” filed Dec. 3, 2020, which claims the benefit of U.S. Provisional Application No. 62/945,039, entitled “DELIVERY APPARATUS AND METHODS FOR IMPLANTING PROSTHETIC HEART VALVES,” filed on Dec. 6, 2019, all of which are incorporated by reference herein in their entirety.

### FIELD

The present disclosure relates to implantable, mechanically-expandable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically-expandable” prosthetic heart valves. Mechanically-expandable prosthetic heart valves can provide one or more advantages over self-expandable and balloon-expandable prosthetic heart valves. For example, mechanically-expandable prosthetic heart valves can be expanded to various diameters. Mechanically-expandable prosthetic heart valves can also be compressed after an initial expansion (e.g., for repositioning and/or retrieval).

Despite these advantages, mechanically-expandable prosthetic heart valves can present several challenges. For example, it can be difficult to control the forces applied to the prosthetic heart valve and/or the delivery apparatus during the implantation procedure. These difficulties can be compounded when the delivery apparatus is disposed in a tortuous pathway, such as a patient's vasculature. It can also be difficult to release a mechanically-expandable prosthetic heart valve from the delivery apparatus. Additionally, given the number of moving components to control, typical delivery apparatus can be difficult and/or time-consuming for a user to operate. Accordingly, there is a need for improved delivery apparatus and methods for implanting mechanically-expandable prosthetic heart valves.

### SUMMARY

Described herein are prosthetic heart valves, delivery apparatus, and methods for implanting prosthetic heart valves. The disclosed delivery apparatus and methods can, for example, help to ensure that the forces applied to the prosthetic heart valve by the delivery apparatus are evenly distributed. This can reduce the likelihood that the delivery apparatus and/or the prosthetic heart valve will become damaged during the implantation procedure. The disclosed delivery apparatus and methods can also help to ensure that the prosthetic heart valve is uniformly expanded. The delivery apparatus disclosed herein are also relatively simple and/or easy to use. This can, for example, reduce the risk of mistakes and/or reduce the time it takes to implant a prosthetic heart valve.

In one representative embodiment, a delivery apparatus for implanting a prosthetic heart valve is provided. The delivery apparatus includes a handle, a first shaft, a plurality of actuation shafts, and a control mechanism. The first shaft has a first end portion, a second end portion, and one or more lumens extending from the first end portion to the second end portion. The first end portion is coupled to the handle. The actuation shafts each have a proximal end portion and a distal end portion, and the actuation shafts extend through the one or more lumens of the first shaft. The control mechanism is coupled to the actuation shafts and to the handle. The control mechanism includes a first mode of operation and a second mode of operation. In the first mode of operation, the proximal end portions of the actuation shafts can move axially relative to each other and relative to the first shaft, and in the second mode of operation, the actuation shafts can be moved axially simultaneously.

In some embodiments, the delivery apparatus is a part of a delivery assembly that also includes a mechanically-expandable prosthetic heart valve.

In another representative embodiment, a delivery apparatus includes a handle, a first shaft, a plurality of actuation shafts, and a force control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The force control mechanism is coupled to the actuation shafts and to the handle. The force control mechanism is configured such that the proximal end portions of the actuation shafts can move axially relative to each other when the first shaft is curved.

In some embodiments, the force control mechanism includes a pulley system interconnecting the actuation shafts.

In another representative embodiment, a delivery apparatus includes a handle, a first shaft, a plurality of actuation shafts, and a displacement control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The displacement control mechanism is coupled to the actuation shafts and to the handle. The displacement control mechanism is configured such that the proximal end portions of the actuation shafts can move axially relative to each other when the first shaft is curved.

In some embodiments, the displacement control mechanism comprises one or more gear assemblies.

In another representative embodiment, a delivery apparatus includes a handle, a first shaft, and a plurality of actuation shafts. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft.

In another representative embodiment, a delivery apparatus includes a handle, a first shaft, a plurality of actuation shafts, a force control mechanism, and a displacement control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The force control mechanism is coupled to the actuation shafts and to the handle. The force control mechanism is configured such that the proximal end portions of the actuation shafts can move axially relative to each other when the first shaft is curved. The displacement control mechanism is coupled to the actuation shafts and to the handle. The displacement control mechanism is configured such that the proximal end portions of the actuation shafts can move axially relative to each other when the first shaft is curved.

In another representative embodiment, a delivery apparatus includes a handle, a first shaft, a plurality of actuation shafts, and a force control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The force control mechanism is coupled to the actuation shafts and configured to evenly distribute forces applied to the actuation shafts.

In another representative embodiment, a delivery apparatus includes a handle, a first shaft, a plurality of actuation shafts, and a displacement control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The displacement control mechanism is coupled to the actuation shafts and configured such that the proximal end portions of the actuation shafts can move axially relative to each other when the first shaft is curved.

In another representative embodiment, a delivery apparatus includes a handle, a first shaft, a plurality of actuation shafts, a force control mechanism, and a displacement control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The force control mechanism is coupled to the actuation shafts and configured to evenly distribute forces applied to the actuation shafts. The displacement control mechanism is coupled to the actuation shafts and configured such that the proximal end portions of the actuation shafts can move axially relative to each other when the first shaft is curved.

In another representative embodiment, a force control mechanism for a delivery apparatus for implanting a prosthetic heart valve is provided. The force control mechanism includes a pulley system and a movable carriage. The pulley system is configured for interconnecting a plurality of actuation shafts of a delivery apparatus. The movable carriage is connected to the pulley system and is configured to be movably coupled to a handle of a delivery apparatus. The pulley system and the movable carriage are configured to move axially and/or rotationally to balance forces applied to and/or carried by the actuation shafts of the delivery apparatus.

In another representative embodiment, a force control mechanism for a delivery apparatus for implanting a prosthetic heart valve is provided. The force control mechanism includes a first pulley, a second pulley, a third pulley, and a carriage. The first pulley is configured to be coupled to first and second actuation shafts of a delivery apparatus. The second pulley is configured to be coupled to a third actuation shaft of the delivery apparatus. The third pulley is configured to be coupled to the third actuation shaft of the delivery apparatus. The carriage is configured to be movably coupled to a handle of the delivery apparatus. The first and second pulleys are rotatably coupled to the carriage, and the carriage is axially movable relative to the third pulley. Proximal end portions of the first and second actuation shafts move axially relative to each other and the first pulley rotates when tension in the first and second actuation shafts is uneven. A proximal end portion of the third actuation shaft moves axially relative to the first and second actuation shafts and the second and third pulleys rotate when tension in the third actuation shaft and the first or second actuation shafts is uneven.

In another representative embodiment, a displacement control mechanism for a delivery apparatus configured for implanting a prosthetic heart valve is provided. The displacement control mechanism includes one or more gear assemblies. The gear assemblies are configured to be coupled to actuation shafts of a delivery apparatus. The gear assemblies are configured to allow proximal end portions of the actuation shafts to move independently relative to each other in an axial direction, and configured to rotate the actuation shafts simultaneously about their respective axes.

In another representative embodiment, a shaft for a delivery apparatus configured for implanting a prosthetic heart valve is provided. The shaft includes a plurality of helical lumens extending from a first end portion of the shaft to a second end portion of the shaft, and each helical lumen is configured to receive an actuation shaft of a delivery apparatus.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a delivery assembly comprising a mechanically-expandable prosthetic heart valve and a delivery apparatus.

FIG. 2 is a perspective view of the prosthetic heart valve.

FIG. 3 is another perspective view of the prosthetic heart valve without the valve structure and with the frame of the prosthetic heart valve in a radially expanded configuration.

FIG. 4 is a side view of the prosthetic heart valve in a radially compressed configuration.

FIG. 5 is a detail of an actuator of the prosthetic heart valve.

FIG. 6 is a cross-sectional view of the actuator of the prosthetic heart valve.

FIG. 7 is a side view of a proximal end portion of the delivery apparatus.

FIG. 8 is a side view of a distal end portion of the delivery apparatus.

FIG. 9 is a cross-sectional view of shafts of the delivery apparatus, taken along the line 9-9 as shown in FIG. 8.

FIG. 10 is a detail view of distal end portions of shafts of the delivery apparatus.

FIG. 11 is a detail view of the prosthetic heart valve released from the delivery apparatus.

FIG. 12 is a detail view of the prosthetic heart valve coupled to the delivery apparatus.

FIG. 13 is a side view of the prosthetic heart valve coupled to the delivery apparatus with the prosthetic heart valve in the radially expanded configuration.

FIG. 14 is a side view of the prosthetic heart valve coupled to the delivery apparatus with the prosthetic heart valve in the radially compressed configuration.

FIG. 15 is a side view of the distal end portion of the delivery assembly.

FIGS. 16-19 depict an exemplary implantation procedure in which the prosthetic heart valve is implanted in a heart (shown in cross-section) with the delivery apparatus.

FIG. 20 is a schematic view of a handle of the delivery apparatus comprising an exemplary force control mechanism.

FIG. 21 is a schematic view of another handle of the delivery apparatus comprising another exemplary force control mechanism.

FIG. 22 is a schematic view of a handle of the delivery apparatus comprising a force control mechanism, according to another embodiment.

FIG. 23 is a side view of the delivery apparatus comprising an exemplary displacement control mechanism.

FIG. 24 is a perspective view of an exemplary coupling member of the displacement control mechanism of FIG. 23.

FIG. 25 is a detail view of the distal end portion of the displacement control mechanism of FIG. 23, showing the coupling member in a distal position.

FIG. 26 is a detail view of the distal end portion of the displacement control mechanism of FIG. 23, showing the coupling member in a proximal position.

FIGS. 27-28 show various perspective views of an exemplary inner gear of the displacement control mechanism of FIG. 23.

FIG. 29 shows a perspective view an exemplary outer gear of the displacement control mechanism of FIG. 23.

FIG. 30 shows an end view of an exemplary gear assembly of the displacement control mechanism of FIG. 23.

FIG. 31 shows a partial cross sectional view of the gear assembly of the displacement control mechanism of FIG. 23.

FIG. 32 is a side view of the delivery apparatus comprising a displacement control mechanism, according to another embodiment.

FIG. 33 is a detail view of the distal end portion of the displacement control mechanism of FIG. 32.

FIG. 34 is a cross-sectional view showing the distal end portion of the displacement control mechanism of FIG. 32.

FIG. 35 is a perspective view of the proximal end portion of the delivery apparatus comprising a displacement control mechanism, according to another embodiment.

FIG. 36 is a perspective view of an exemplary first gear assembly of the displacement control mechanism of FIG. 35.

FIGS. 37-39 show various perspective views of exemplary components of the first gear assembly of the displacement control mechanism of FIG. 35.

FIG. 40 is a perspective view of an exemplary second gear assembly of the displacement control mechanism of FIG. 35.

FIG. 41 is a perspective view of an exemplary slidable outer gear and the displacement control mechanism of FIG. 35, showing the outer gear in a proximal position.

FIG. 42 is a perspective view of the slidable outer gear and the displacement control mechanism of FIG. 35, showing the outer gear in a distal position.

FIG. 43 is a top view of the proximal end portion of the delivery apparatus comprising another exemplary displacement control mechanism.

FIG. 44 is an end view of an exemplary first gear assembly of the displacement control mechanism of FIG. 43.

FIG. 45 is an end view of an exemplary second gear assembly of the displacement control mechanism of FIG. 43.

FIG. 46 is a partial cross-sectional view of the first gear assembly of the displacement control mechanism of FIG. 43, showing the first gear assembly in an unlocked configuration.

FIG. 47 is a partial cross-sectional view of the first gear assembly of the displacement control mechanism of FIG. 43, showing the first gear assembly in a locked configuration.

FIG. 48 is a side view of an exemplary shaft for the delivery apparatus.

FIGS. 49-51 are various cross-sectional views of the shaft of FIG. 48.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Examples of the Disclosed Technology

Described herein are prosthetic heart valves, delivery apparatus, and methods for implanting prosthetic heart valves. The disclosed delivery apparatus and methods can, for example, help to ensure that the forces applied to the prosthetic heart valve by the delivery apparatus are evenly distributed. This can reduce the likelihood that the delivery apparatus and/or the prosthetic heart valve will become damaged during the implantation procedure. The disclosed delivery apparatus and methods can also help to ensure that the prosthetic heart valve is uniformly expanded. The delivery apparatus disclosed herein are also relatively simple and/or easy to use. This can, for example, reduce the risk of mistakes and/or reduce the time it takes to implant a prosthetic heart valve.

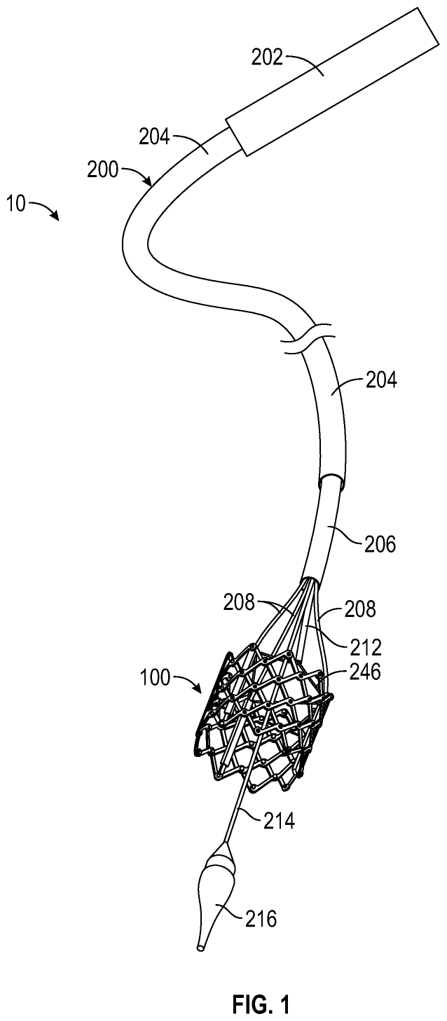

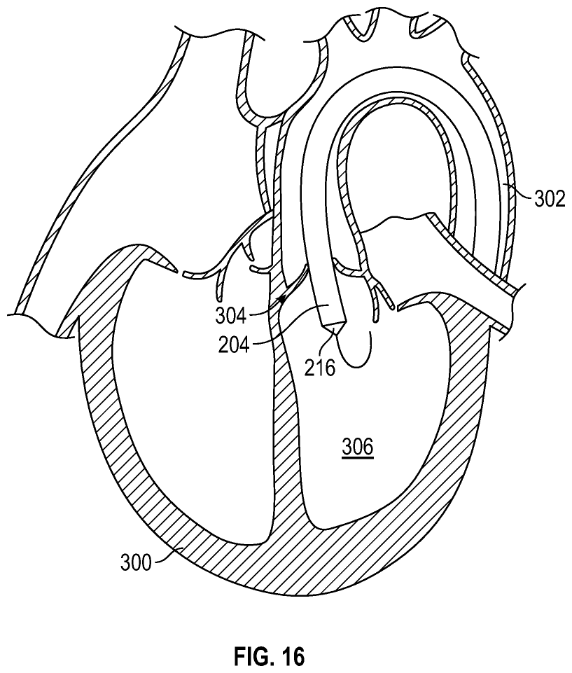

FIG. 1 shows a delivery assembly 10, according to one embodiment. In the illustrated embodiment, the delivery assembly 10 comprises a prosthetic heart valve 100 and a delivery apparatus 200. The prosthetic valve 100 can be configured to replace a native heart valve (e.g., aortic, mitral, pulmonary, and/or tricuspid valves). As shown, the prosthetic valve 100 can be releasably coupled to a distal end portion of the delivery apparatus 200. The delivery apparatus 200 can be used to deliver and implant the prosthetic valve 100 in the native heart valve of a patient (see, e.g., FIGS. 16-19). Additional details regarding the prosthetic valve 100 and the delivery apparatus 200 are provided below.

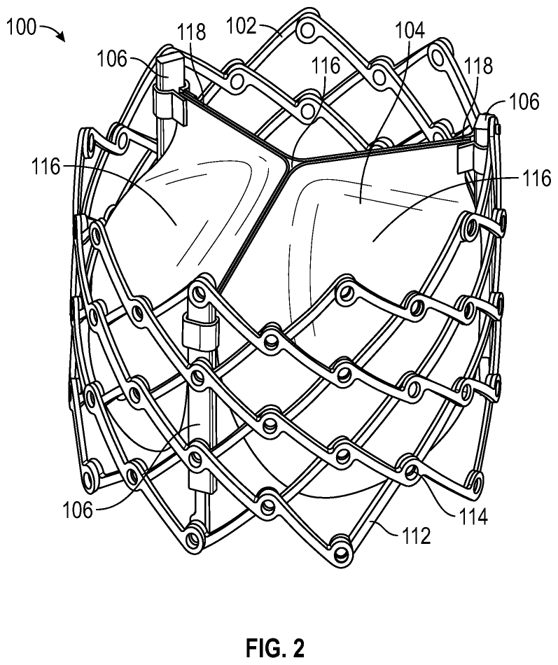

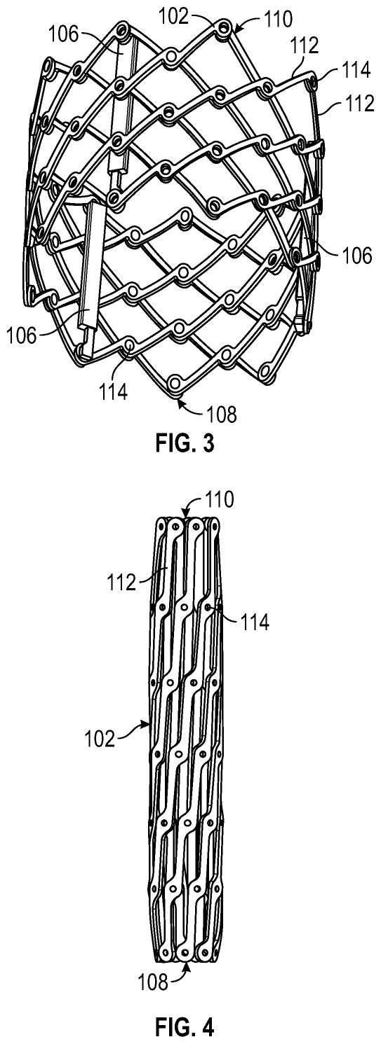

FIG. 2 shows the prosthetic valve 100. As shown, the prosthetic valve 100 comprises three main components: a frame 102, a valve structure 104, and one or more actuators 106 (e.g., three actuators in the illustrated embodiment). The frame 102 (which can also be referred to as “a stent” or “a support structure”) can be configured for supporting the valve structure 104 and for securing the prosthetic valve 100 within a native heart valve. The valve structure 104 is coupled to the frame 102 and/or to the actuators 106. The valve structure 104 is configured to allow blood flow through the prosthetic valve 100 in one direction (i.e., antegrade) and to restrict blood flow through the prosthetic valve 100 in the opposition direction (i.e., retrograde). The actuators 106 are coupled to the frame 102 and are configured to adjust expansion of the frame 102 to a plurality of configurations including one or more functional or expanded configurations (e.g., FIGS. 2-3), one or more delivery or compressed configurations (e.g., FIG. 4), and/or one or more intermediate configurations between the functional and delivery configurations. It should be noted that the valve structure 104 of the prosthetic valve 100 is not shown FIGS. 1 and 3-4 for purposes of illustration.

Referring to FIG. 3, the frame 102 of the prosthetic valve 100 has a first end 108 and a second end 110. In the illustrated embodiment, the first end 108 of the frame 102 is an inflow end and the second end 110 of the frame 102 is an outflow end. In other embodiments, the first end 108 of the frame 102 can be the outflow end and the second end 110 of the frame 102 can be the inflow end.

The frame 102 can be made of any of various suitable materials, including biocompatible metals and/or biocompatible polymers. Exemplary biocompatible metals from which the frame can be formed include stainless steel, cobalt chromium alloy, and/or nickel titanium alloy (which can also be referred to as “NiTi” or “nitinol”).

Referring still to FIG. 3, the frame 102 includes a plurality of interconnected struts 112 arranged in a lattice-type pattern. In FIG. 3, the frame 102 of the prosthetic valve 100 is in a radially expanded configuration, which results in the struts 112 of the frame 102 extending diagonally relative to a longitudinal axis of the prosthetic valve 100. In other configurations, the struts 112 of the frame 102 can be offset by a different amount than the amount depicted in FIG. 3. For example, FIG. 4 shows the frame 102 of the prosthetic valve 100 in a radially compressed configuration. In this configuration, the struts 112 of the frame 102 extend parallel (or at least substantially parallel) to the longitudinal axis of the prosthetic valve 100.

To facilitate movement between the expanded and compressed configurations, the struts 112 of the frame 102 are pivotably coupled to one another at one or more pivot joints along the length of each strut. For example, each of the struts 112 can be formed with apertures at opposing ends and along the length of the strut. The frame 102 comprises hinges at the locations where struts 112 overlap and are pivotably coupled together via fasteners such as rivets or pins 114 that extend through the apertures of the struts 112. The hinges allow the struts 112 to pivot relative to one another as the frame 102 moves between the radially expanded and the radially compressed configurations, such as during assembly, preparation, and/or implantation of the prosthetic valve 100.

In some embodiments, the frame 102 can be constructed by forming individual components (e.g., the struts 112 and pins 114 of the frame 102) and then mechanically assembling and coupling the individual components together. In other embodiments, the struts are not coupled to each other with respective hinges but are otherwise pivotable or bendable relative to each other to permit radial expansion and contraction of the frame. For example, a frame can be formed (e.g., via laser cutting, electroforming or physical vapor deposition) from a single piece of material (e.g., a metal tube). Further details regarding the construction of frames and prosthetic valves are described in U.S. Pat. Nos. 10,603,165 and 10,806,573, U.S. Publication Nos. 2018/0344456, and International Application Nos. PCT/US2019/056865 and PCT/US2020/040318, which are incorporated by reference herein. Additional examples of expandable prosthetic valves that can be used with the delivery apparatus disclosed herein are described in U.S. Pat. Nos. 9,700,442 and 9,827,093, which are incorporated by reference herein.

Referring again to FIG. 2, the valve structure 104 of the prosthetic valve 100 is coupled to the frame 102. The valve structure 104 is configured to allow blood flow through the prosthetic valve 100 from the inflow end 108 to the outflow end 110 and to restrict blood from through the prosthetic valve 100 from the outflow end 110 to the inflow end 108. The valve structure 104 can include, for example, a leaflet assembly comprising one or more leaflets 116 (e.g., three leaflets in the illustrated embodiment).

The leaflets 116 of the prosthetic valve 100 can be made of a flexible material. For example, the leaflets 116 of the leaflet assembly can be made from in whole or part, biological material, bio-compatible synthetic materials, or other such materials. Suitable biological material can include, for example, bovine pericardium (or pericardium from other sources).

Referring to FIG. 2, the leaflets 116 can be arranged to form commissures 118 (e.g., pairs of adjacent leaflets), which can, for example, be mounted to respective actuators 106. Further details regarding prosthetic heart valves, including the manner in which the valve structure 104 can be coupled to the frame 102 of the prosthetic valve 100, can be found in U.S. Pat. Nos. 6,730,118, 7,393,360, 7,510,575, 7,993,394, and 8,652,202, and U.S. Publication No. 2018/0325665, which are incorporated by reference herein.

The valve structure 104 can be coupled to the actuators 106. For example, the commissures 118 of the valve structure 104 can be coupled to the housing members 122 of the actuators 106. Additional details regarding coupling the valve structure to the actuators can be found, for example, in International Application No. PCT/US2020/040318.

As shown in FIG. 3, the actuators 106 of the prosthetic valve 100 are mounted to and spaced circumferentially around the inner surface of the frame 102. The actuators 106 are configured to, among other things, radially expand and/or radially compress the frame 102. For this reason, the actuators 106 can also be referred to as “expansion mechanisms.” The actuators 106 are also configured to lock the frame 102 at a desired expanded configuration. Accordingly, the actuators 106 can be referred to as “lockers” or “locking mechanisms.” Each of the actuators 106 can be configured to form a releasable connection with one or more respective actuation shafts of a delivery apparatus, as further described below.

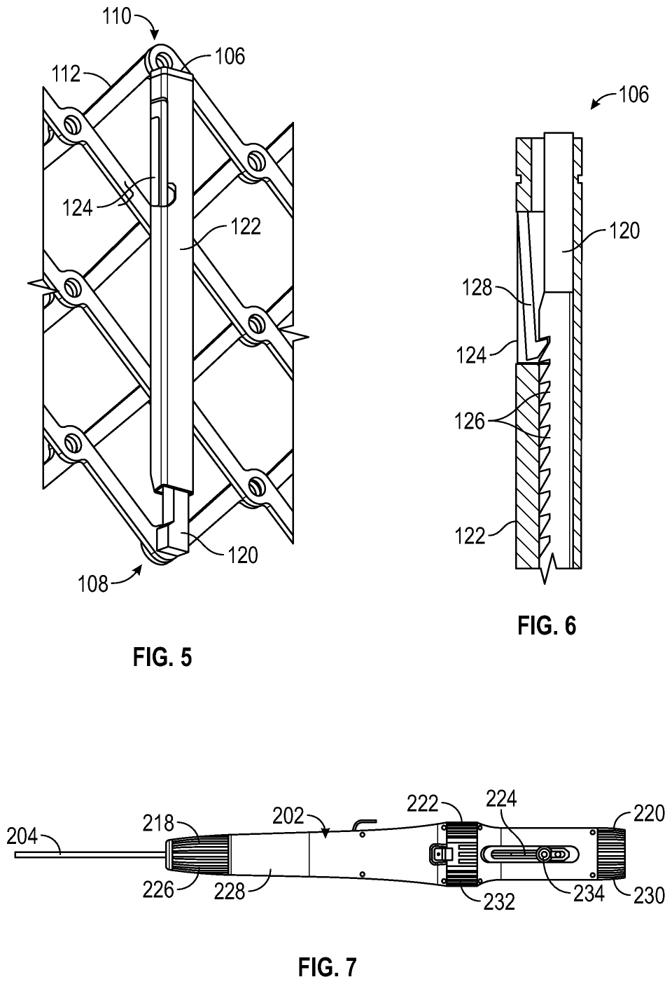

Referring now to FIGS. 5-6, each actuator 106 comprises a rack member 120 (which can also be referred to as an “actuation member”), a housing member 122 (which can also be referred to as a “support member”), and a locking member 124. The rack members 120 can be coupled to the frame 102 of the prosthetic valve 100 at a first axial location (e.g., toward the inflow end 108 of the frame 102), and the housing members 122 can be coupled to the frame at a second axial location (e.g., toward the outflow end 110 of the frame 102). The rack members 120 extend through and are axially movable relative to respective housing members 122. Thus, relative axial movement between the rack members 120 and the housing members 122 applies axial directed forces to the frame 102 and results in radial expansion/compression of the frame 102 as the struts 112 of the frame 102 pivot relative to each other about the pins 114. Moving the rack members 120 proximally (e.g., up in the orientation depicted in FIGS. 5-6) relative to the housing members 122 radially expands the frame 102 (e.g., FIG. 3). Conversely, moving the rack members 120 distally (e.g., down in the orientation depicted in FIGS. 5-6) relative to the housing members 122 radially compresses the frame 102 (e.g., FIG. 4).

As shown in FIG. 6, one or more of the rack members 120 includes a segment with a plurality of teeth 126. Each locking member 124 is coupled to a respective housing member 122 and comprises a pawl 128 biased to engage the teeth 126 of the rack member 120. In this manner, the rack member 120 and the locking member 124 form a ratchet-type mechanism that allows the rack member 120 to move proximally relative to the housing member 122 (thereby allowing expansion of the prosthetic valve 100) and that restricts the rack member 120 from moving distally relative to the housing member 122 (thereby restricting compression of the prosthetic valve 100).

In the illustrated embodiment, the locking member 124 is integrally formed with the housing member 122 as a unitary structure. In other embodiments, the locking member 124 and the housing member 122 can be formed as separate components that are coupled together (e.g., with fasteners, adhesive, welding, and/or other means for coupling).

In the illustrated embodiment, the prosthetic valve 100 includes three actuators 106. In other embodiments, a greater or fewer number of actuators can be used. For example, in one embodiment, the prosthetic valve can have one actuator. As another example, the prosthetic valve can have two actuators. In yet another example, a prosthetic valve can have 4-15 actuators.

Although not shown, the prosthetic valve 100 can also include one or more skirts or sealing members. For example, the prosthetic valve 100 can include an inner skirt mounted on the inner surface of the frame 102. The inner skirt can function as a sealing member to prevent or decrease perivalvular leakage, to anchor the leaflets 116 to the frame 102, and/or to protect the leaflets 116 against damage caused by contact with the frame 102 during crimping and during working cycles of the prosthetic valve 100. The prosthetic valve 100 can also include an outer skirt mounted on the outer surface of the frame 102. The outer skirt can function as a sealing member for the prosthetic valve by sealing against the tissue of the native valve annulus and thus reducing paravalvular leakage around the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

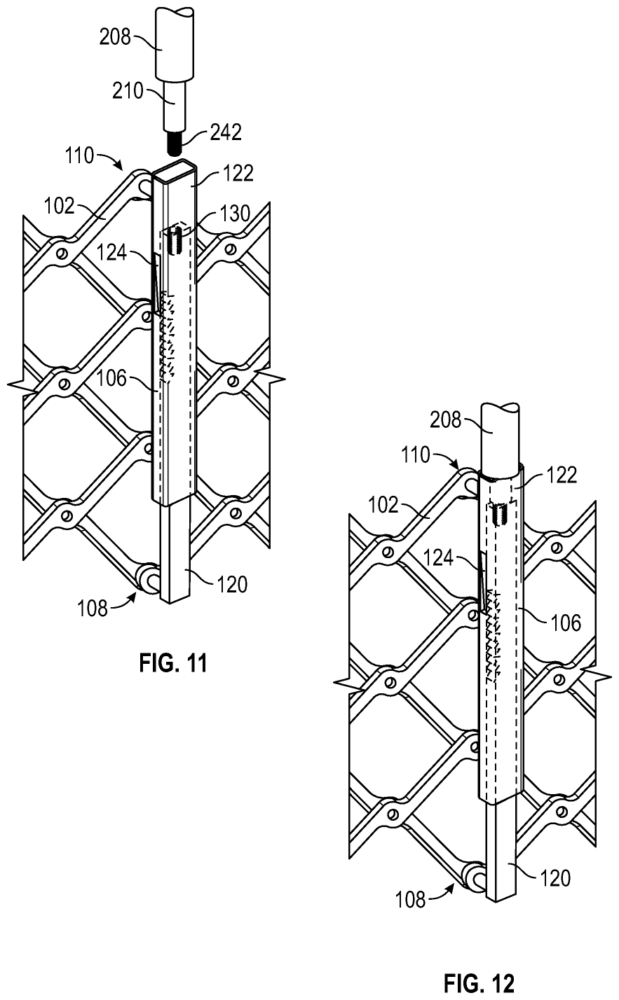

FIGS. 7-10 show the delivery apparatus 200 and its components, which can also be referred to as a “valve catheter” or a “delivery catheter.” As shown, the delivery apparatus 200 comprises a handle 202, a first shaft 204, a second shaft 206, one or more support sleeves 208 (e.g., three in the illustrated embodiment), one or more actuation shafts 210 (e.g., three in the illustrated embodiment), an optional recompression shaft 212, a nosecone shaft 214, and a nosecone 216. The handle 202 is configured for manipulating the shafts and sleeves relative to each other. The prosthetic heart valve 100 can be releasably coupled to the distal end portion of the delivery apparatus 200 (see, e.g., FIGS. 11-13), and the delivery apparatus 200 can be used for positioning the prosthetic valve 100, and/or for expanding, compressing, and locking the prosthetic valve 100 in a desired radially expanded configuration.

In the illustrated embodiment, the delivery apparatus 200 comprises three pairs of a support sleeve 208 and an actuation shaft 210 (i.e., one pair of a support sleeve 208 and an actuation shaft 210 for each actuator 106 of the prosthetic valve 100). In other embodiments, the delivery apparatus 200 can comprise less than three (e.g., 1-2) or more than three (e.g., 4-15) pairs of support sleeves 208 and actuation shafts 210, depending on the number of actuators a prosthetic valve includes.

The handle 202 of the delivery apparatus 200 comprises one or more mechanisms configured to move the shafts and sleeves relative to each other. For example, as shown in FIG. 7, the handle 202 comprises a first mechanism 218, a second mechanism 220, a third mechanism 222, and/or a fourth mechanism 224.

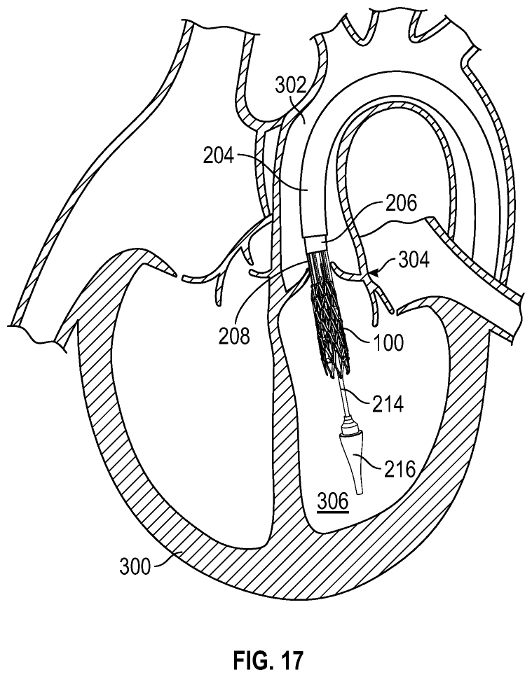

The first mechanism 218 of the handle 202 is coupled to the first and second shafts 204, 206 and is configured to move the first and second shafts 204, 206 axially relative to each other. As further explained below, the first mechanism 218 of the handle 202 can be used to deploy the prosthetic valve 100 from the delivery capsule of the first shaft 204 (see FIG. 17). As such, the first mechanism 218 can be referred to as “a deployment mechanism.”

In the illustrated embodiment, the first mechanism 218 includes a first knob 226 configured for actuating the first mechanism 218. Although not shown, in other embodiments, the first mechanism 218 can comprise various other types of actuators configured for actuating the first mechanism 218, such as buttons, switches, etc. The first mechanism 218 can also include one or more other non-illustrated components (such as electric motors, rotatable shafts, drive screws, gear assemblies, etc.) configured to facilitate and/or restrict relative axial movement between the first and second shafts 204, 206. For example, the first mechanism 218 can be configured such that rotating the first knob 226 (and/or an electric motor) relative to a housing 228 of the handle 202 results in relative axial movement between the first and second shafts 204, 206.

The second mechanism 220 of the handle 202 is coupled to the actuation shafts 210 and is configured to move the actuation shafts 210 axially relative to the support sleeves 208. When the prosthetic valve 100 is coupled to the delivery apparatus 200 via the actuation shafts 210, the second mechanism 220 of the handle 202 can be used to radially expand and/or compress the prosthetic valve 100, as further explained below. Accordingly, the second mechanism 220 can be referred to as “an actuation mechanism” and/or “an expansion mechanism.”

In the illustrated embodiment, the second mechanism 220 comprises a second knob 230 configured for actuating the second mechanism 220. In other embodiments, the second mechanism 220 can comprise various other types of actuators. Although not shown, the second mechanism 220 can also include one or more additional components configured to facilitate and/or restrict relative axial movement of the actuation shafts 210 relative to the support sleeves 208. For example, the second mechanism 220 can comprise electric motors, drive screws, gear assemblies, and/or other components. In some embodiments, the second mechanism 220 can be configured such that rotating the second knob 230 (and/or an electric motor) relative to the housing 228 of the handle results in relative axial movement between the actuation shafts 210 and the support sleeves 208.

The third mechanism 222 of the handle 202 is also coupled to the actuation shafts 210 and is configured to rotate the actuation shafts 210 relative to the support sleeves 208. In this manner, the third mechanism 222 can be used to simultaneously couple and release the actuation shafts 210 to/from the prosthetic valve 100, as further described below. Thus, the third mechanism 222 can be referred to as “a release mechanism” or “a coupling mechanism.”

In the illustrated embodiment, the third mechanism 222 comprises a third knob 232 configured for actuating the third mechanism 222. In other embodiments, the third mechanism 222 can comprise various other types of actuators. The third mechanism 222 can also comprise one or more other components (e.g., a gear assembly and/or an electric motor) configured to facilitate and/or restrict relative rotational movement between the actuation shafts 210 and the support sleeves 208. For example, the third mechanism 222 can be configured such that rotating the third knob 232 relative to the housing 228 results in rotation of the actuation shafts 210 relative to the support sleeves 208.

The fourth mechanism 224 of the handle 202 is coupled to the nosecone shaft 214 and is configured to move the nosecone shaft 214 and the nosecone 216 axially relative to the first and second shafts 204, 206. As such, the fourth mechanism 224 can be referred to as a “nosecone mechanism.”

In the illustrated embodiment, the fourth mechanism 224 comprises a slider 234 configured for actuating the fourth mechanism 224. Although not shown, the fourth mechanism 224 can comprise various other components configured to facilitate and/or restrict relative axial movement of the nosecone shaft 214 and the first and second shafts 204, 206. For example, in some embodiments, the fourth mechanism 224 can comprise one or more biasing members (e.g., springs) configured to bias the nosecone shaft 214 to a pre-determined axial position relative to the first and second shafts 204, 206. In such embodiments, the slider 234 can be biased to a particular axial position relative to the housing 228 (e.g., to a proximal position). The nosecone shaft 214 can be moved axially relative to the first and second shafts 204, 206 by sliding the slider 234 relative to the housing 228 with sufficient force to overcome the opposing force of the biasing members. Upon release, the slider 234 can return to the biased position. In other embodiments, the fourth mechanism can comprise a rotatable knob, an electric motor, and/or a drive screw configured to convert relative rotational movement between the knob (and/or motor) and the housing into relative axial movement between the nosecone shaft and the first and second shafts.

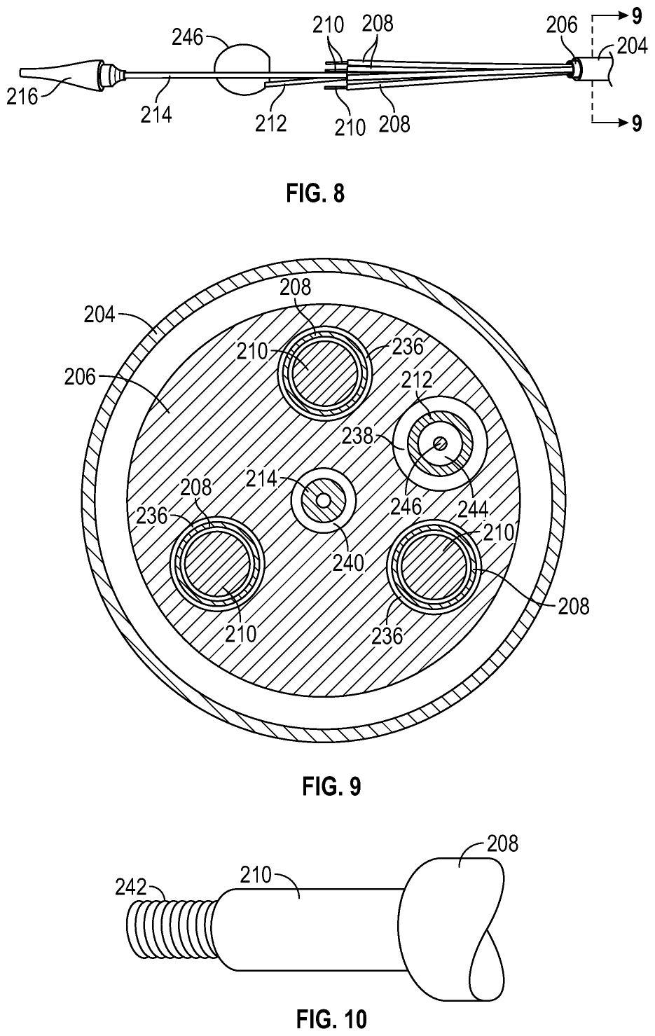

Referring now to FIGS. 7-8, a proximal end portion of the first shaft 204 is coupled to and extends distally from the handle 202. The first shaft 204 comprises a lumen for housing the second shaft 206 of the delivery apparatus 200. The distal end portion of the first shaft 204 is configured to receive the prosthetic valve 100 in the radially compressed configuration (see FIGS. 14-17). As such, the first shaft 204 can be referred to as “a sheath” or “a delivery capsule”. Alternatively, the delivery capsule can be a separately formed component coupled to the distal end portion of the first shaft 204.

As shown in FIGS. 8-9, the second shaft 206 extends coaxially through and is axially movable relative to the first shaft 204. The second shaft 206 can comprise a plurality of lumens extending axially therethrough and can thus be referred to as “a multi-lumen shaft.” For example, as shown in FIG. 9, the second shaft 206 includes one or more first lumens 236 (e.g., three in the illustrated embodiment) spaced circumferentially relative to each other. The first lumens 236 can be configured to receive respective actuation shafts 210 and/or support sleeves 208. In the illustrated embodiment, the first lumens 236 are evenly spaced relative to each other (i.e., spaced apart by about 120 degrees). In other embodiments, the first lumens 236 can be non-evenly spaced relative to each other.

In some embodiments, the second shaft 206 can also include one or more additional lumens. For example, as shown in FIG. 9, the second shaft 206 includes a recompression lumen 238 and a guidewire lumen 240. The guidewire lumen 240 can be radially centrally disposed in the second shaft 206. The recompression lumen 238 can be disposed radially outwardly relative to the guidewire lumen 240. In some embodiments, the recompression lumen 238 can be radially aligned with and/or spaced circumferentially relative to the first lumens 236.

The support sleeves 208 can extend distally from respective first lumens 236 of the second shaft 206 and can be configured to contact the actuators 106 of the prosthetic valve 100 (see FIG. 12). The support sleeves 208 can be relatively more rigid than the actuation shafts 210. As such, the support sleeves 208 can be used to apply distally-directed forces to the housing members 122 of the actuators 106, which can oppose proximally-directed forces applied to the rack members 120 of the actuators 106 by the actuation shafts 210 of the delivery apparatus 200, thereby enabling expansion of the prosthetic valve 100 caused by relative axial movement between the rack members 120 and the housing members 122 of the actuators 106.

In the illustrated embodiment, the support sleeves 208 are relative short tubes that are coupled to the distal end portion of the second shaft 206 but do not extend all the way through the second shaft 206 to the handle 202. The sleeves 208 can, in some instances, be secured to the inner surfaces of the second shaft 206 that define the first lumens 236 (e.g., via adhesive). In some embodiments, proximal end portions of the support sleeves 208 can be coupled to the handle 202, and the support sleeves 208 can extend through respective first lumens 236 of the second shaft 206 and beyond the distal end of the second shaft 206. In either instance, each of the support sleeves 208 comprises a lumen configured to receive a respective actuation shaft 210, as shown in FIG. 9.

The actuation shafts 210 can extend distally from the handle 202, through respective first lumens 236 of the second shaft 206, and through the lumens of respective support sleeves 208. The distal end portions of the actuation shafts 210 can comprise mating features configured to releasably couple the actuation shafts to the actuators 106 of the prosthetic valve 100. For example, as shown in FIGS. 10-12, the distal end portions of the actuation shafts 210 comprise external threads 242 configured to mate with corresponding internal threads 130 of the rack member 120 of the actuators 106.

In some embodiments, the actuation shafts 210 can be relatively flexible members. For example, the actuation shafts can be wires, cables, cords, sutures, etc. In other embodiments, the actuation shafts can be relatively rigid members, such as a rod. In other embodiments, the actuation shafts 210 can comprise one or more relatively flexible segments (e.g., at the distal end portions) and one or more relatively rigid segments (e.g., at the proximal end portions).

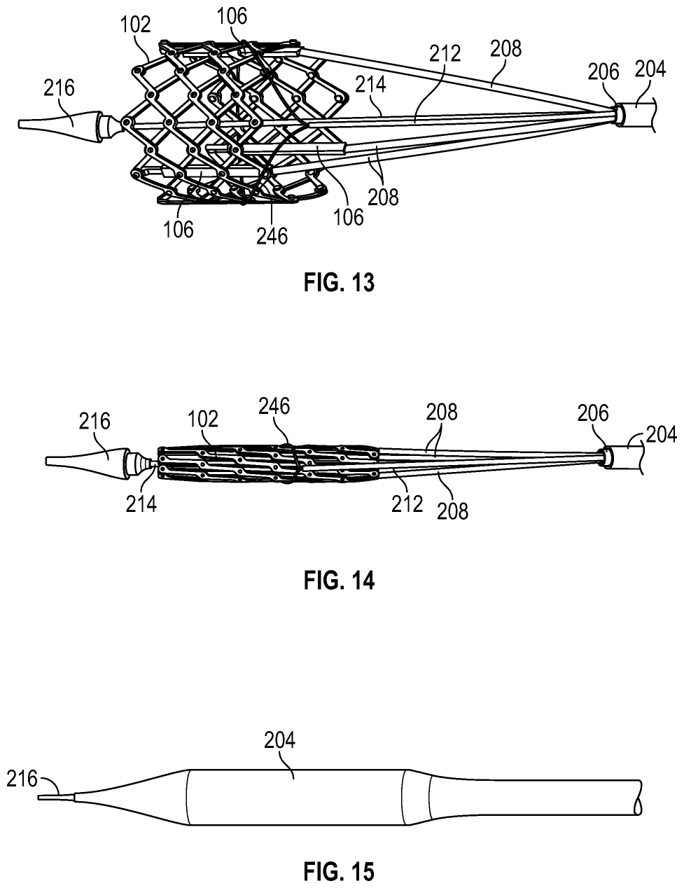

Referring to FIG. 8, the recompression shaft 212 extends from the handle 202 through the recompression lumen 238 of the second shaft 206. As shown in FIG. 9, the recompression shaft 212 comprises a lumen 244 through which a recompression member 246 (e.g., wire, cable, suture, etc.) extends. As shown in FIG. 13, the recompression member 246 can extend around the prosthetic valve 100 in a lasso-like manner. As such, the recompression member 246 can be used to recompress the prosthetic valve 100 by tensioning and thus constricting the recompression member 246 around the prosthetic valve 100.

The prosthetic valve 100 can be coupled to a distal end portion of the delivery apparatus 200 to form the delivery assembly (see FIGS. 11-13), and the delivery apparatus 200 can be used to implant the prosthetic valve 100 within a patient's body (see FIGS. 13-19). The prosthetic valve 100 can be coupled to the delivery apparatus 200 by positioning the delivery apparatus 200 in the configuration shown in FIG. 8. With the prosthetic valve 100 in the radially expanded configuration, the prosthetic valve 100 can be positioned over a proximal portion of the nosecone 216 and the nosecone shaft 214 and optionally within the loop of the recompression member 246, as shown in FIG. 13. The actuators 106 of the prosthetic valve 100 can be positioned adjacent the distal ends of the actuation shafts 210, as shown in FIG. 11. The actuation shafts 210 can then be inserted into the housing members 122 of the actuators 106 and threadably coupled to the rack members 120 of the actuators 106, as shown in FIG. 12.

With the prosthetic valve 100 releasably coupled to the delivery apparatus 200 (see FIG. 13), the prosthetic valve 100 can be radially compressed by actuating the actuators 106, by tensioning the recompression member 246, and/or by inserting the prosthetic valve 100 and delivery apparatus 200 into a crimping device. Additional details about an exemplary crimping device for mechanically-expandable prosthetic valves can be found in International Application No. PCT/US2020/042141, which is incorporated by reference herein. FIG. 14 shows the prosthetic valve 100 in a radially compressed configuration. The first shaft 204 of the delivery apparatus 200 can then be advanced over the second shaft 206 of the delivery apparatus 200 and the prosthetic valve 100 such that the prosthetic valve 100 is disposed within the lumen of the first shaft 204 and the distal end of the first shaft 204 abuts the nosecone 216, as shown in FIG. 15. This can be accomplished, for example, by actuating the first mechanism 218 of the handle 202.

The distal end portion of the delivery assembly 10 can then be inserted into a patient's vasculature, and the prosthetic valve 100 can be advanced to an implantation location using the delivery apparatus 200. For example, FIGS. 16-19 show an exemplary implantation procedure for implanting the prosthetic valve 100 within a patient's heart 300 using a transfemoral delivery procedure. In other embodiments, various other delivery procedures can be used, such as transventricular, transapical, transseptal, etc.

Referring to FIG. 16, the distal end portion of the delivery assembly 10 is inserted into a patient's vasculature such that the first shaft 204 extends through the patient's aorta 302 and such that the nosecone 216 extends through the patient's native aortic annulus 304 and into the left ventricle 306 of the patient's heart 300. Turning to FIG. 17, the prosthetic valve 100 can be deployed from the first shaft 204 of the delivery apparatus 200 by actuating the first mechanism 218 of the handle 202, which moves the first shaft 204 of the delivery apparatus 200 proximally relative to the second shaft 206 of the delivery apparatus 200 (and/or moves the second shaft 206 distally relative to the first shaft 204). The first shaft 204 can be moved further proximally such that the support sleeves 208 are exposed from the distal end of the first shaft 204 (see, e.g., FIG. 14).

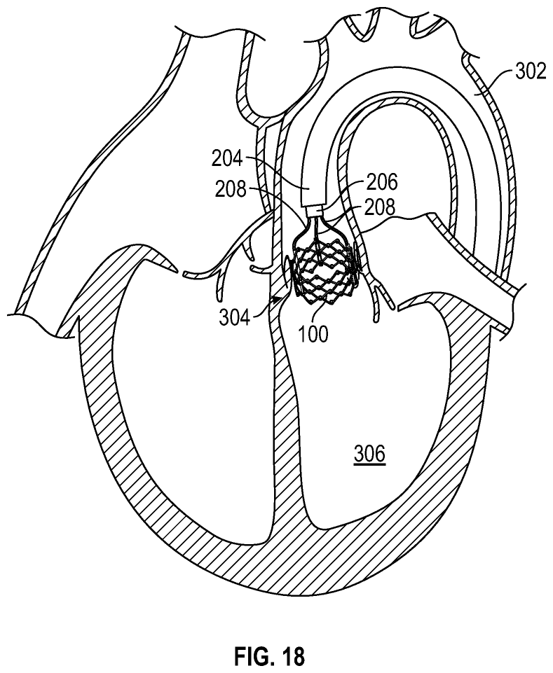

As shown in FIG. 18, the prosthetic valve 100 can then be radially expanded. This can be accomplished, for example, by actuating the second mechanism 220 of the handle 202 such that the actuation shafts 210 and the rack members 120 of the actuators 106 (which are coupled to the actuation shafts 210) move proximally relative to the support sleeves 208 and the housing members 122 of the actuators 106 (which abut the distal ends of the support sleeves 208). When the prosthetic valve 100 is desirably positioned and secured within the native aortic annulus 304, the locking members 124 can engage the rack members 120 to retain the prosthetic valve 100 in the expanded state.

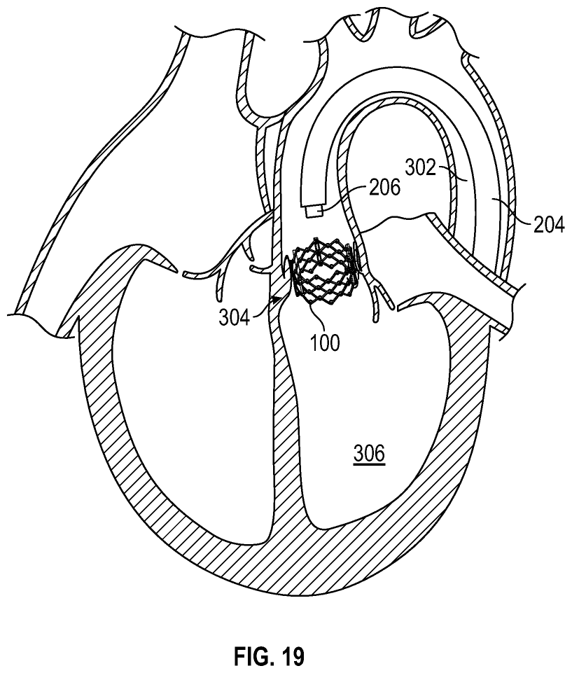

If re-positioning of the prosthetic valve is desired, the second mechanism 202 can be used to actuate the actuators 106 to radially compress the prosthetic valve 100. In lieu of or in addition to using the second mechanism 202, the prosthetic valve 100 can be recompressed and repositioned and/or retrieved using the recompression member 246. In some instances, the recompression member 246 can radially compress the prosthetic valve to a diameter that is smaller than is possible using only the actuators 106. It should be noted that, for purposes of illustration, the recompression shaft 212 and the recompression member 246 are not shown in FIGS. 17-18, and that the nosecone shaft 214 and the nosecone 216 are not shown in FIGS. 18-19.

Once expanded and secured, the prosthetic valve 100 can then be released from the delivery apparatus 200, as shown in FIG. 19. This can be accomplished by actuating the third mechanism 222 of the handle 202. This rotates the actuation shafts 210 of the delivery apparatus 200 relative to the rack members 120 of the prosthetic valve 100, thereby de-coupling the threads 242 of the actuation shafts 210 from the threads 130 of the rack members 120. The actuation shafts 210, the support sleeves 208, and the second shaft 206 can then be withdrawn into the first shaft 204, and the delivery apparatus 200 can be removed from the patient's body.

During an implantation procedure, a delivery apparatus is advanced through a patient's vasculature. The patient's vasculature can include various curves, including some relative sharp curves (e.g., a native aortic arch (see FIGS. 16-19)). When the delivery apparatus is curved, some of the shafts of the delivery apparatus travel different path lengths than other shafts of the delivery apparatus. The length of a shaft's path can vary according to its radial distance from the neutral axis. For example, for the delivery apparatus 200, the central longitudinal axis of the first and second shafts 204, 206 forms a neutral axis. As such, the first and second shafts 204, 206 travel the same length when they extend around a curve because the first and second shafts 204, 206 are coaxial and concentric. As shown in FIG. 9, the actuation shafts 210 are spaced radially outwardly from the central longitudinal axis of the first and second shafts 204, 206. In other words, the actuation shafts 210 are non-coaxial and eccentric with the first and second shafts 204, 206. As such, when the delivery apparatus 200 is disposed around a curve, each of the actuation shafts 210 travels a different path length. When the actuation shafts 210 are all the same length, the different path lengths can produce uneven tension in the actuation shafts 210 and/or cause the actuation shafts 210 to stretch. Uneven tension and/or stretching in the actuation shafts 210 is undesirable because it can cause non-uniform force distribution across the actuation shafts and/or non-uniform displacement of the actuation shafts. Non-uniform forces in the actuation cables can result in excessive force in one or more of the actuation shafts 210, which can, in some instances, damage the actuators 106 and/or actuation shafts 210. Non-uniform displacement of the actuation cables can, for example, result in non-uniform radial expansion of the prosthetic heart valve. Accordingly, it is desirable to reduce or prevent non-uniform forces and/or non-uniform displacement in the actuation shafts.

Disclosed herein are various control mechanisms and multi-lumen shafts configured for controlling the forces and/or displacement of the actuation shafts, even when the actuation shafts are curved. These control mechanisms can, in some instances, be coupled to an expansion mechanism and/or release mechanism of a delivery apparatus. The disclosed control mechanisms can, for example, help to evenly distribute the load on the actuation shafts. Additionally or alternatively, the disclosed control mechanisms can also adjust the lengths of the actuation shafts relative to each other so that the prosthetic valve evenly expands upon actuation of the expansion mechanism. The control mechanisms disclosed herein can be used, for example, with the delivery apparatus 200.

Generally speaking, the disclosed control mechanisms operate by allowing one end of the actuation shafts (e.g., the proximal end portions) to move relative to the other components of the delivery apparatus rather than being fixed at both ends. In this manner, the actuation shafts can “float” as the delivery apparatus is curved, thereby preventing uneven tension and/or stretching in the actuation shafts.

In some embodiments, a control mechanism can be a force control mechanism for a delivery apparatus. A force control mechanism can be configured to evenly distribute the forces applied to the actuation shafts of a delivery apparatus. In some embodiments, a force control mechanism can comprise a pulley system. A pulley system can include one or more pulleys interconnecting the actuation shafts. The pulleys can allow the proximal end portions of the actuation shafts to move relative to each other to evenly distribute the loads of the actuation shafts. A force control mechanism can, in some embodiments, be coupled to the actuation mechanism of a delivery apparatus.

In some embodiments, a control mechanism can be a displacement control mechanism for a delivery apparatus. In particular embodiments, a displacement control mechanism can comprise one or more gear assemblies coupled to the actuation shafts of a delivery apparatus. The gear assemblies can be configured to move the actuation shafts axially and/or rotationally relative to other components of the delivery apparatus and/or a prosthetic heart valve. In this manner, a displacement mechanism can be used, for example, to expand a prosthetic heart valve and/or release a prosthetic heart valve from a delivery apparatus. In particular embodiments, a displacement control mechanism can be coupled to an actuation mechanism and/or a release mechanism of a delivery apparatus.

In other embodiments, a multi-lumen shaft comprising a plurality of helical lumens can be provided. The helical lumens can be configured for receiving respective actuation shafts of the delivery apparatus. The helical lumens can, for example, help to ensure that the actuation shafts travel the same or at substantially the same distance, even with the multi-lumen shaft is curved. Accordingly, the multi-lumen shafts disclosed herein can, for example, help to ensure uniform valve expansion.

In certain instances, a delivery apparatus can have a force control mechanism, a displacement control mechanism, and/or a multi-lumen shaft with helical lumens. In other instances, a delivery apparatus can include a force control mechanism and omit a displacement control mechanism and/or a multi-lumen shaft with helical lumens. In yet other embodiments, a delivery apparatus can comprise various other combinations and/or sub-combinations of force control mechanisms, displacement control mechanisms, and/or multi-lumen shafts with helical lumens.

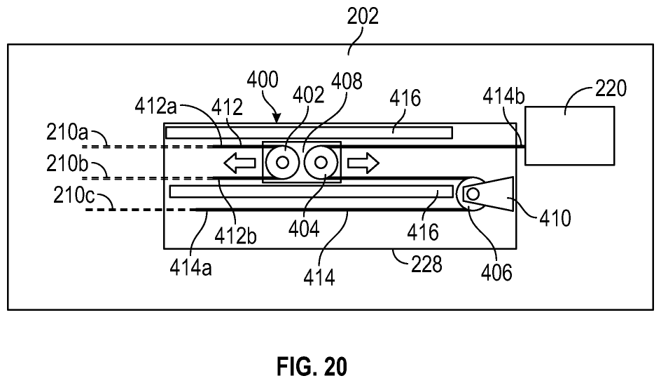

FIG. 20 shows a force control mechanism 400, according to one embodiment. As shown, the force control mechanism 400 can, in some instances, be a component of the delivery apparatus 200. In some of those instances, the force control mechanism can, for example, be housed within the handle 202 of the delivery apparatus 200. The force control mechanism 400 can be coupled to and disposed between the actuation shafts 210 and the actuation mechanism 220. In this manner, the force control mechanism 400 can be used to evenly distribute forces in and/or applied to the actuation shafts 210.

The force control mechanism 400 comprises a plurality of pulleys coupled to the actuation shafts 210 and the actuation mechanism 220. One or more of the pulleys can be disposed on a movable carriage such that the carriage and pulleys can move relative to the housing 228 of the handle 202, and one or more of the pulleys can be coupled to the housing 228 of the handle 202 such that the pulleys are stationary relative to the housing 228.

More specifically, the force control mechanism 400 comprises a first dynamic pulley 402, a second dynamic pulley 404, a stationary pulley 406, a carriage 408, and a base member 410. The first and second dynamic pulleys 402, 404 are rotatably coupled to the carriage 408. The stationary pulley 406 is rotatably coupled to the base member 410, which is fixedly coupled to the housing 228 of the handle 202.

In the illustrated embodiment, the force control mechanism 400 also comprises a first connecting member 412 and a second connecting member 414. The first and second connecting members 412, 414 can be a flexible cord, wire, cable, suture, etc. The first connecting member 412 extends around the first dynamic pulley 402 and has a first end portion 412a coupled to a proximal end portion of a first actuation shaft 210a and a second end portion 412b coupled to a proximal end portion of a second actuation shaft 210b. The second connecting member 414 extends around the second dynamic pulley 404 and the stationary pulley 406 and has a first end portion 414a coupled to a proximal end portion of a third actuation shaft 210c and a second end portion 414b coupled to the actuation mechanism 220.

In other embodiments, a force control mechanism can omit the connecting members. In such embodiments, the first and second actuation shafts 210a, 210b be can be integrally formed or directly coupled together. Also, the third actuation shaft 210c can be directly coupled to the actuation mechanism 220.

The carriage 408 is axially movable relative to the housing 228 of the handle 202. For example, the carriage 408 can be slidably coupled to the housing 228 such that the carriage 408 can move axially relative to the housing 228. In some embodiments, the carriage 408 can be coupled to the housing 228 via tracks 416 configured to facilitate relative axial movement between the carriage 408 and the housing 228. In some instances, friction reducing elements (e.g., bearings, wheels, rollers, lubrication, lubricous materials, etc.) can be disposed between the carriage 408, the tracks 416, and/or the housing 228 to help the carriage 408 move more easily relative to the tracks 416 and/or the housing 228.

In operation, the proximal end portions of the first and second actuation shafts 210a, 210b can freely move axially relative to each other via the first connecting member 412 and the first dynamic pulley 402. As such, any difference in force (e.g., tension) between the first and second actuation shafts 210a, 210b will be balanced as the proximal end portions of the first and second actuation shafts 210a, 210b move axially relative to each other. Also, the proximal end portion of the third actuation shaft 210c can freely move axially relative to the proximal end portions of the first and/or second actuation shafts 210a, 210b via the second connecting member 414, the second dynamic pulley 404, the stationary pulley 406, and the carriage 408. As such, any difference in force between the third actuation shaft 210c and the first and/or second actuation shafts 210a, 210b will be balanced as the proximal end portions of the actuation shafts 210 move axially relative to each other.

Also, when the actuation mechanism 220 is actuated to expand the prosthetic valve 100 and tension increases in the second connecting member 414, the force control mechanism 400 evenly distributes the tension in the second connecting member 414 across the actuation shafts 210 by allowing the proximal end portions of the actuation shafts 210 to move axially relative to each other. For example, as shown in FIG. 20, the proximal end portions of each of the actuation shafts 210 are in different axial locations relative to the handle 202.

Even force distribution across the actuation shafts can help to ensure that no one actuation shaft bears excessive load, which can result in uneven expansion of the prosthetic valve and/or damage to the actuation shafts (e.g., damage to the threads 242 at the distal end portions of the actuation shafts 210). As a result, the force control mechanism can, for example, improve the functionality, safety, and/or reliability of the delivery apparatus.

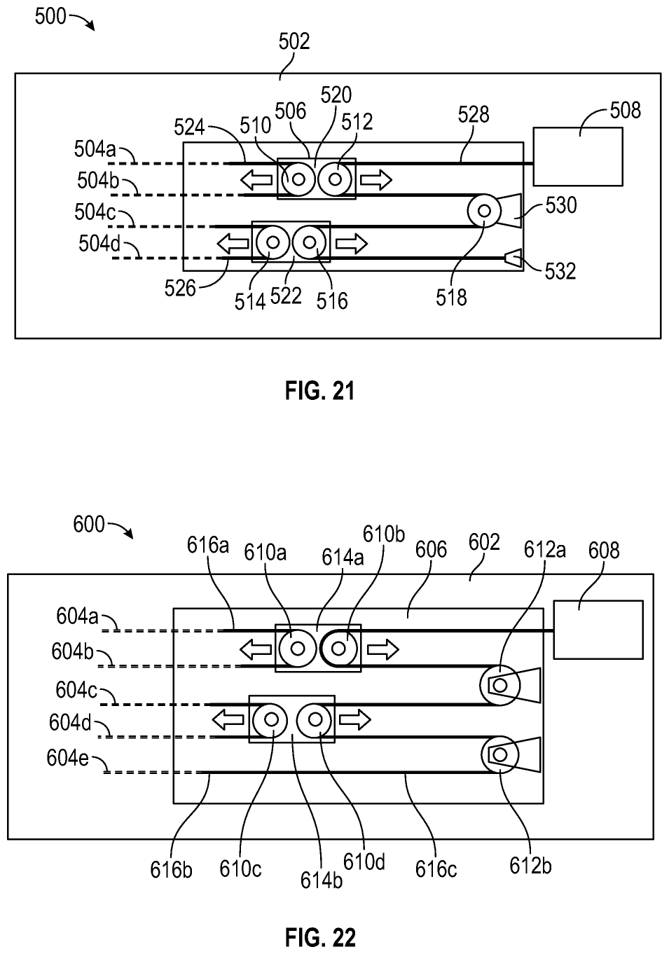

FIG. 21 shows a portion of a delivery apparatus 500, according to another embodiment. The delivery apparatus 500 comprises a handle 502 and a plurality of actuation shafts 504a-504d (collectively or generically, “the actuation shafts 504”). The delivery apparatus 500 also comprises a force control mechanism 506 and an actuation mechanism 508. The actuation shafts 504 are coupled to the handle 502 via the force control mechanism 506 and the actuation mechanism 508. The force control mechanism 506 and the actuation mechanism 508 are configured generally similar to the force control mechanism 400 and the actuation mechanism 220, respectively, except that the force control mechanism 506 is configured to balance the forces of four actuation shafts rather than three actuation shafts.

In the illustrated embodiment, the force control mechanism 506 of the delivery apparatus 500 comprises a first dynamic pulley 510, a second dynamic pulley 512, a third dynamic pulley 514, a fourth dynamic pulley 516, a stationary pulley 518, a first carriage 520, and a second carriage 522, a first connecting member 524, a second connecting member 526, a third connecting member 528, a base member 530, and an anchor 532. The first and second dynamic pulleys 510, 512 are rotatably mounted to the first carriage 520, which is movably coupled to the handle 502. The third and fourth dynamic pulleys 514, 516 are rotatably mounted to the second carriage 522, which is also movably coupled to the handle 502. The stationary pulley 518 is rotatably mounted to the base member 530, which is fixedly coupled to the handle 502. The first connecting member 524 extends around the first dynamic pulley 510 and has a first end portion coupled to the proximal end portion of the first actuation shaft 504a and a second end portion coupled to the proximal end portion of the second actuation shaft 504b. The second connecting member 526 extends around the third dynamic pulley 514 and has a first end portion coupled to the proximal end portion of the third actuation shaft 504c and a second end portion coupled to the proximal end portion of the fourth actuation shaft 504d. The third connecting member 528 extends from the actuation mechanism 508, around the second dynamic pulley 512, around the stationary pulley 518, around the fourth dynamic pulley 516, and to the anchor 532. The anchor 532 is fixedly coupled to the handle 502.

The first connecting member 524 and the first dynamic pulley 510 allow the proximal end portions of the first and second actuation shafts 504a, 504b to move axially relative to each other. This distributes forces evenly between the first and second actuation shafts 504a, 504b. The second connecting member 526 and the third dynamic pulley 514 allow the proximal end portions of the third and fourth actuation shafts 504c, 504d to move axially relative to each other. This distributes forces evenly between the third and fourth actuation shafts 504c, 504d. The third connecting member 528, the second and fourth dynamic pulleys 512, 516, the stationary pulley 518, and the anchor 532 allow the first and second carriages 520, 522 to move axially relative to each other, which in turn allows the proximal end portions of the first and second actuation shafts 504a, 504b to move axially relative to the proximal end portions of the third and fourth actuation shafts 504c, 504d. This distributes forces evenly between all of the actuation shafts 504.

In other embodiments, the force control mechanism 506 can omit the connecting members and the actuation shafts can be directly coupled together and/or to other components of the delivery apparatus 500.

FIG. 22 shows a portion of a delivery apparatus 600, according to another embodiment. The delivery apparatus 600 comprises a handle 602 and a plurality of actuation shafts 604a-604e (collectively or generically, “the actuation shafts 604”). The delivery apparatus 600 also comprises a force control mechanism 606 and an actuation mechanism 608, and the actuation shafts 604 are coupled to the handle 602 via the force control mechanism 606 and the actuation mechanism 608. The force control mechanism 606 and the actuation mechanism 608 are configured generally similar to the force control mechanism 400 and the actuation mechanism 220, respectively, except that the force control mechanism 606 is configured to balance the forces of five actuation shafts rather than three.

The force control mechanism 606 comprises a plurality of dynamic pulleys 610 (e.g., four in the illustrated embodiment (610a-610d)), a plurality of static pulleys 612 (e.g., two in the illustrated embodiment (612a-612b)), a plurality of carriages 614 (e.g., two in the illustrated embodiment (614a-614b)), and a plurality of connecting members 616 (e.g., three in the illustrated embodiment (616a-616c).

The components of the force control mechanism 606 cooperate to allow the proximal end portions of the actuation shafts 604 to move axially relative to each other in a manner similar to that described above with respect to the force control mechanisms 400 and 506. This results in forces being evenly distributed across the actuation shafts 604.

The force control mechanisms 400, 506, and 606 are configured for delivery apparatus having three, four, or five actuation shafts, respectively. In other embodiments, the force control mechanisms can be configured for use with delivery apparatus having fewer than three (e.g., two) or more than five (e.g., 6-15) actuation shafts.

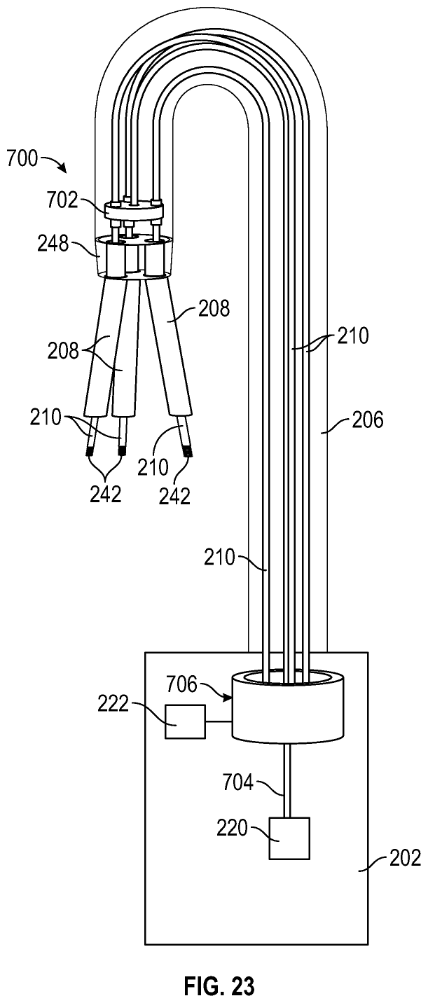

FIG. 23 shows a displacement control mechanism 700. As shown, the displacement control mechanism 700 can, in some instances, be used with the delivery apparatus 200. The displacement control mechanism 700, among other things, allows all of the actuation shafts 210 to be simultaneously moved axially (e.g., to expand a prosthetic valve). The displacement control mechanism 700 also allows simultaneous release of all of the actuation shafts (e.g., when de-coupling a prosthetic valve from the delivery apparatus). The displacement control mechanism 700 additionally allows the proximal end portions of the actuation shafts of the delivery apparatus to move axially relative to each other in the event the actuation shafts travel different path lengths (e.g., when the actuation shafts bend around a curve).

In the illustrated embodiment, the displacement control mechanism 700 comprises three main components: a coupling member 702, an actuation member 704, and a gear assembly 706. The coupling member 702 of the displacement control mechanism 700 is disposed toward the distal end portion of the shaft 206 of the delivery apparatus 200 and is coupled to the actuation shafts 210 of the delivery apparatus 200. It should be noted that the shaft 206 is shown as transparent for purposes of illustration. The actuation member 704 of the displacement control mechanism 700 extends through the shaft 206 and is coupled to the coupling member 702 of the displacement control mechanism 700 at its distal end portion and is coupled to the actuation mechanism 220 of the delivery apparatus 200 at its proximal end portion. The gear assembly 706 of the displacement control mechanism 700 is disposed within the handle 202 of the delivery apparatus 200 and is coupled to the proximal end portions of the actuation shafts 210 and to the release mechanism 222 of the delivery apparatus 200. In this manner, axial movement of the actuation member 704 relative to the shaft 206 moves the coupling member 702 and the actuation shafts 210 axially (e.g., to expand a prosthetic valve), and rotational movement of the gear assembly 706 relative to the shaft 206 rotates the actuation shafts 210 (e.g., to release a prosthetic valve from the delivery apparatus 200). Additional details regarding the displacement control mechanism 700 and its components are provided below.

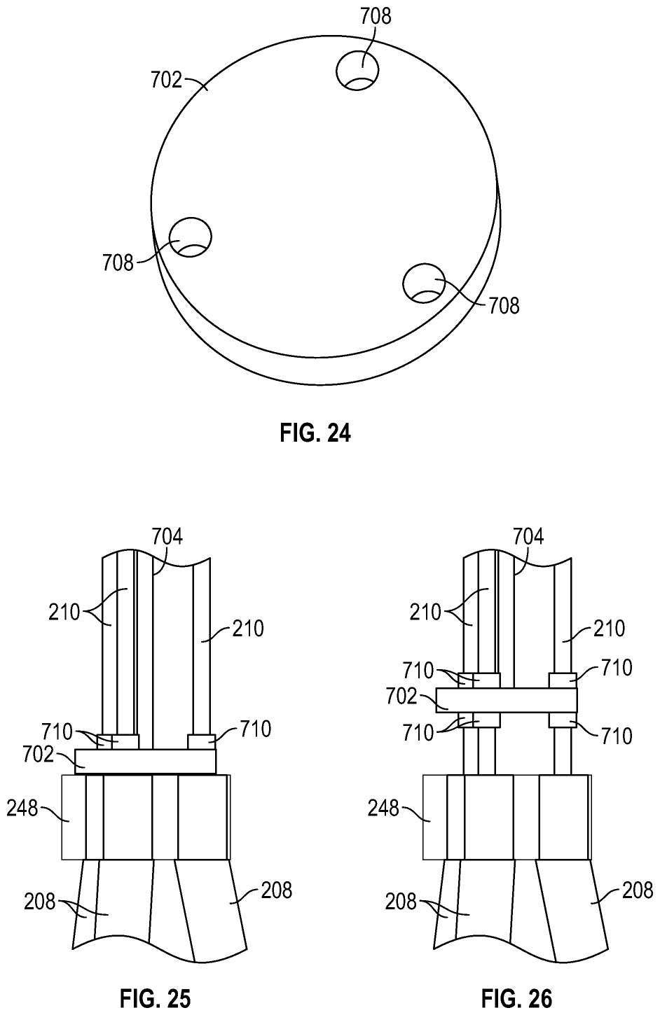

Referring to FIG. 24, the coupling member 702 of the displacement control mechanism 700 comprises a cylindrical or disc shape. In other embodiments, the coupling member can comprise various other shapes (e.g., cube, prism, etc.).

The coupling member 702 comprises a plurality of openings 708 extending axially therethrough. As shown in FIG. 26, the openings 708 of the coupling member 702 are configured such that the actuation shafts 210 can extend through and rotate freely relative to the coupling member 702.

Referring to FIG. 26, to restrict relative axial movement between the coupling member 702 and the actuation shafts 210, a plurality of stopper members 710 are provided. The stopper members 710 are fixedly coupled to the actuation shafts 210 (e.g., with fasteners, adhesive, welding, frictional engagement, etc.) at locations adjacent the proximal and distal facing surfaces of the coupling member 702. The stopper members 710 are radially larger than the openings 708 of the coupling member 702. As a result, the stopper members 710 abut the proximal and distal facing surfaces of the coupling member 702 and thus restrict relative axial movement between the actuation shafts 210 and the coupling member 702.

As shown in FIGS. 25-26, the distal end portion of the actuation member 704 is coupled to the coupling member 702. Accordingly, axial movement of the actuation member 704 results in axial movement of the coupling member 702 and thus the actuation shafts 210. For example, FIG. 25 shows the actuation member 704, the coupling member 702, and the actuation shafts 210 in a proximal position in which the coupling member 702 abuts a distal manifold 248 of the delivery apparatus 200, which is shown as transparent for purposes of illustration. The manifold 248 of the delivery apparatus 200 is coupled to the distal end portion of the shaft 206 and is used to couple the support sleeves 208 to the shaft 206. The manifold 248 also acts as a distal stopper for the coupling member 702.

The actuation member 704 can be coupled to the coupling member 702 in various ways including knotting, fasteners, adhesive, embedding, etc. Although not shown, in some embodiments, the coupling member 702 can comprise an attachment element (e.g., a bore, opening, eyelet, etc.) configured to facilitate attachment of the actuation member 704 to the coupling member 702.

As shown schematically in FIG. 23, the proximal end portion of the actuation member 704 is coupled to the actuation mechanism 220 of the handle 202. In some embodiments, the actuation mechanism 220 can comprise a spool or other apparatus configured for gathering and releasing the actuation member 704, which can be used to increase and decrease the tension of the actuation member 704. The actuation mechanism 220 can comprise a first operating mode which increases tension in the actuation member 704 and thus moves the actuation member 704, the coupling member 702, and the actuation shafts 210 proximally relative to the support sleeves 208. As such, the first operating mode can be used, for example, to radially expand a prosthetic valve (e.g., the prosthetic valve 100) coupled to the distal end portions of the actuation shafts 210. The actuation mechanism 220 can comprise a second operating mode which decreases tension in the actuation member 704 and moves (or allows) the actuation member 704, the coupling member 702, and the actuation shafts 210 to move distally. Accordingly, the second operating mode can be used, for example, to radially compress a prosthetic valve (e.g., the prosthetic valve 100) that is coupled to the distal end portions of the actuation shafts 210. In this manner, the displacement control mechanism 700 advantageously allows for simultaneous axial movement of all of the actuation shafts 210, which in turn provides simultaneous actuation of the actuators 106 of the prosthetic valve 100. This can, for example, improve uniform expansion of the prosthetic valve.

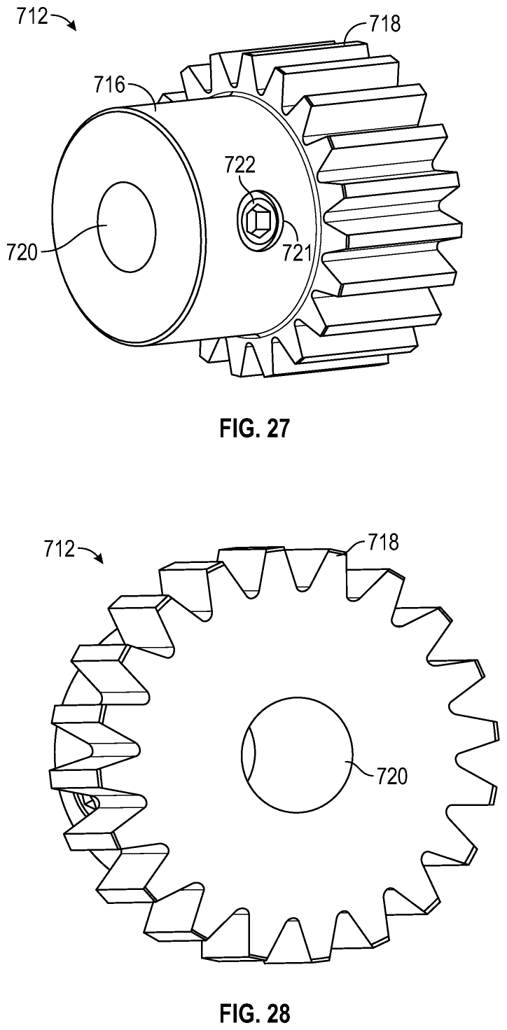

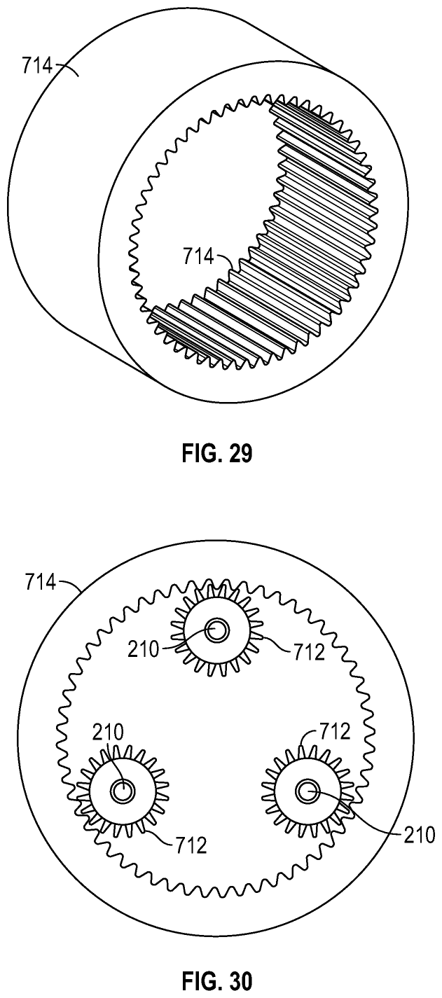

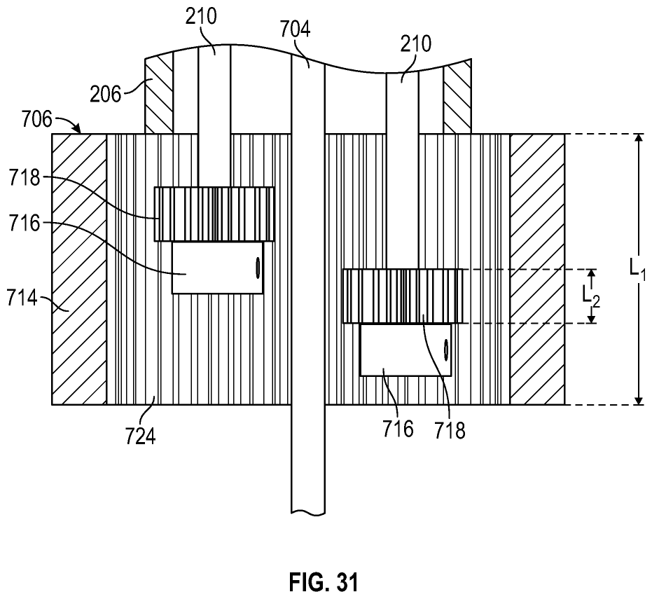

FIGS. 27-31 show the gear assembly 706 of the displacement control mechanism 700 and its components. Referring initially to FIGS. 30 and 31, the gear assembly 706 comprises a plurality of inner gears 712 and an outer gear 714 circumscribing the inner gears 712. The inner gears 712 are coupled to the proximal end portions of the actuation shafts 210. The inner gears 712 and the proximal end portions of the actuation shafts 210 can move axially relative to the outer gear 714. The outer gear 714 engages each of the inner gears 712 such that rotating the outer gear 714 about its central longitudinal axis results in the inner gears 712 rotating about their respective longitudinal axes. In this manner, the gear assembly 706 can be used to simultaneously rotate the actuation shafts 210 relative to the shaft 206, e.g., when coupling and/or releasing a prosthetic valve to/from the delivery apparatus 200.

Referring to FIGS. 27-28, the inner gears 712 each comprise an attachment portion 716 and a plurality of teeth 718. The attachment portion 716 can be configured for coupling the inner gear 712 to a corresponding actuation shaft 210 (FIG. 23). For example, in the illustrated embodiment, the attachment portion 716 of the inner gear 712 comprises an axial opening 720 (or a bore) that is configured to receive the proximal end portion of an actuation shaft 210. The attachment portion 716 also comprises a radial opening 721 that intersects the axial opening 720. A securing element 722 (e.g., a set screw) can be disposed in the radial opening 721 and adjustably (e.g., threadably) coupled to the attachment portion 716. Thus, the securing element 722 can extend into the axial opening 720 and contact the actuation shaft 210 to restrict relative movement (e.g., axial and rotational) between the inner gear 712 and the actuation shaft 210. Accordingly, axial movement of the inner gear 712 results in axial movement of the actuation shaft 210, and rotational movement of the inner gear 712 results in rotational movement of the actuation shaft 210.

In lieu of or in addition to the axial opening 720, the radial opening 721, and/or the securing element 722, the inner gears 712 can be secured to the actuation shafts in various other ways. For example, the inner gears 712 can be secured to the actuation shafts 210 via adhesive, welding, and/or other means for coupling. Additionally or alternatively, in some embodiments, each actuation shaft 210 can comprise a “flat” (i.e., a segment with a “D-shaped” cross-sectional profile taken in a plane perpendicular to the longitudinal axis of the actuation shaft). The flat of the actuation shaft can be axially aligned with the radial opening 721 of the inner gear 712 so that the securing element 722 engages the flat of the actuation shaft (rather than a circular portion of the actuation shaft), which provides increased resistance to relative rotational movement between the actuation shaft and the inner gear. Additionally or alternatively, the actuation shaft and the axial opening 720 of the inner gear 712 can comprise corresponding non-circular cross-sectional shapes (e.g., D-shaped, square-shaped, triangle-shaped, star/gear-shaped) which can be mated together and thereby restrict relative rotational movement between the actuation shaft and the inner gear.

The teeth 718 of the inner gear 712 extend radially outwardly from the attachment portion 716. As shown in FIG. 30, the teeth 718 of the inner gears 712 mesh with corresponding radially inwardly facing teeth 724 of the outer gear 714. The inner gears 712 of the displacement control mechanism 700 and the actuation shafts 210 of delivery apparatus 200 can be mounted within the handle 202 of the delivery apparatus 200 such that the inner gears 712 and the actuation shafts 210 can rotate about their respective central axes but cannot move circumferentially (i.e., orbit) relative to the outer gear 714. As such, rotation of the outer gear 714 about its central axis relative to the handle 202 of the delivery apparatus 200 results in rotation of the inner gears 712 and the actuation shafts 210 about their respective central axes relative to the handle 202 (and the shaft 206).

Due to the inner gears 712 having diameters that are smaller than the diameter of the outer gear 714, one revolution of the outer gear 714 about its central axis results in more than one revolution of the inner gears 712 about their respective central axes. Various gear ratios between the inner gears 712 and the outer gear 714 can selected by varying the relative diameters of the inner gears 712 and the outer gear 714.

The inner gears 712 and the actuation shafts 210 can also be mounted within the handle 202 of the delivery apparatus 200 such that the inner gears 712 and the proximal end portions of the actuation shafts 210 can move axially relative to the outer gear 714 and relative to each other. This can advantageously allow the actuation shafts 210 to adjust to various path lengths due to curvature in the shaft 206 (e.g., when curving around the aortic arch). For example, FIG. 31 shows two of the actuation shafts 210 and inner gears 712, each at a different axial position. When the shaft 206 is curved (see, e.g., FIG. 23), a first actuation shaft positioned on an outer portion of the curve travels a longer path length than a second actuation shaft positioned on an inner portion of the curve. Accordingly as shown in FIG. 31, the proximal end portion of the first actuation shaft can move distally relative to the outer gear (and the other actuation shafts and inner gears—assuming the actuation shafts are all the same length), and/or the proximal end portion of the second actuation shaft can move proximally relative to the outer gear (and the other actuation shafts and inner gears). When the shaft 206 is straight, the proximal end portions of the actuation shafts can move axially relative to the outer gear 714 and align axially relative to each other.

To accommodate the axial movement of the proximal end portions of the actuation shafts 210 and the inner gears 712, the outer gear 714 can comprise an axial length L₁ that is greater than an axial length L₂ of the teeth 718 of the inner gears 712. This allows the teeth 718 of the inner gears 712 remain engaged with the teeth 724 of the outer gear 714 as the components move axially relative to each other. The length L₁ of the outer gear 714 can be configured such to allow for a maximum variation in length of the actuation shafts. In other words, the length L₁ of the outer gear 714 relative to the length L₂ of the inner gears 712 is configured such that the teeth 718 of the inner gears 712 remain engaged with the teeth 724 of the outer gear 714 regardless of the axial position of the inner gears 712, which can change based on the degree of curvature of the shaft 206 and/or the circumferential position of the actuation shaft 210 relative to the curve (e.g., as the shaft 206 is torqued). For example, in some embodiments, a ratio of the lengths L₁ and L₂ can be between 1.5-10. In particular embodiments, the ratio of the lengths L₁ and L₂ can be between 2-6. In certain embodiments, the ratio of the lengths L₁ and L₂ can be between 3-5. In yet other embodiments, the ratio of the lengths L₁ and L₂ can be 4-4.5.

The delivery apparatus 200 comprising the displacement control mechanism 700 can be used to implant a prosthetic valve. For example, the prosthetic valve 100 can be coupled to the delivery apparatus 200 such that the actuation shafts 210 of the delivery apparatus 200 are releasably (e.g., threadably) coupled to respective rack members 120 of the prosthetic valve 100 and such that the support sleeves 208 of the delivery apparatus 200 abut respective housing members 122 of the actuators 106, as shown in FIG. 1. The prosthetic valve 100 and the delivery apparatus 200 can be inserted into a patient's body, and the delivery apparatus 200 can be used to deploy and implant the prosthetic valve 100 within the patient's body, similar to the manner described above with respect to FIGS. 16-19. Specifically, as the prosthetic valve 100 and the delivery apparatus 200 are advanced through the patient's vasculature, the shaft 206 can curve through the patient's vasculature to the implantation location. When the shaft 206 curves, the displacement control mechanism 700 allows the proximal end portions of the actuation shafts 210 (and the inner gears 712) to move axially relative to each other and relative to the outer gear 714 to accommodate the different path lengths of the actuation shafts 210. During such movement, the inner gears 712 remain engaged with the outer gears 714.

The prosthetic valve 100 can be expanded by actuating the actuation mechanism 220 of the handle 202, which moves the actuation member 704, the coupling member 702, the actuation shafts 210, and the rack members 120 axially proximally relative to the shaft 206, the support sleeves 208, and the housing members 122. As the actuation member 704 and the actuation shafts 210 move proximally, the inner gears 712 remain engaged with the outer gears 714.

If desired, the prosthetic valve 100 can be recompressed for repositioning and/or retrieval.

Once the prosthetic valve 100 is desirably positioned and secured to within the patient's body, the prosthetic valve 100 can be released from the delivery apparatus 200. This can be accomplished, for example, by actuating the release mechanism 222 of the delivery apparatus 200, which actuates the gear assembly 706 of the displacement control mechanism 700. When the gear assembly 706 is actuated, the outer gear 714 rotates about its central axis and relative to the handle 202, which causes the inner gears 712 to rotate about their respective central axes. It also results in the actuation shafts 210 rotating relative to the rack members 120 of the prosthetic valve 100, which retracts the threads 242 of the actuation shafts 210 from the threads of the rack members 120 and thereby releases the prosthetic valve 100 from the delivery apparatus 200.

Configuring the displacement control mechanism 700 in this manner thus allows a user to simultaneously move multiple actuation shafts (e.g., the actuation shafts 210) axially via a single actuation member (e.g., the actuation member 704). Also, by allowing the proximal end portions of the actuation shafts 210 to move axially relative to each other, the displacement control mechanism 700 ensures that the distal end portions of all of the actuation shafts move a constant (or nearly constant) distance when the actuation member 704 is moved axially. This can, for example, help to ensure that a prosthetic valve is uniformly radially expanded, even when the delivery apparatus is in a curved configuration. The displacement control mechanism 700 can also simplify the actuation mechanism by having a single actuation member. The disclosed displacement control mechanism 700 additionally allows the actuation shafts 210 to be simultaneously rotated via the gear assembly 706. This can, for example, allow a prosthetic valve to be quickly and easily released from the delivery apparatus.

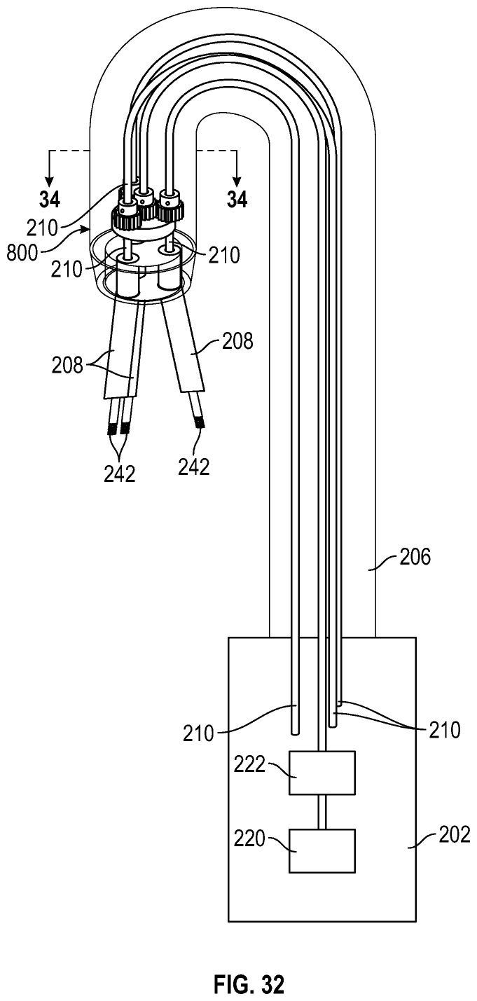

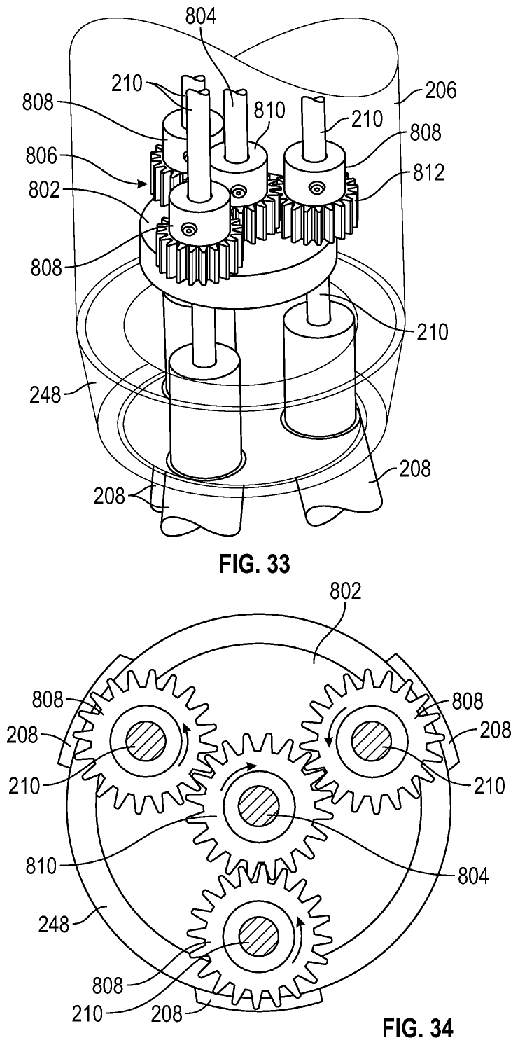

FIGS. 32-34 show a displacement control mechanism 800, according to another embodiment. Referring to FIG. 33, the displacement control mechanism 800 (FIG. 32) comprises a coupling member 802, an actuation member 804, and a gear assembly 806. Generally speaking, the displacement control mechanism 800 is configured and operates similar to the displacement control mechanism 700. One difference between the displacement control mechanism 800 and the displacement control mechanism 700 is that the gear assembly 806 of the displacement control mechanism 800 is disposed at the distal end portion of the delivery apparatus 200 (see FIG. 32) rather than in the handle 202 like the gear assembly 706 of the displacement control mechanism 700 (see FIG. 23). It should be noted that the shaft 206 is omitted from FIG. 34 for purposed of illustration.

The displacement control mechanism 800 can be used with various delivery apparatus. For example, in the illustrated embodiment, the displacement control mechanism 800 is shown with the delivery apparatus 200. Referring to FIG. 32, the coupling member 802 of the displacement control mechanism 800 is disposed within the distal end portion of the shaft 206 of the delivery apparatus 200. For purposes of illustration, the shaft 206 and the manifold 248 are shown as transparent. The coupling member 802 of the displacement control mechanism 800 is coupled to the actuation shafts 210 of the delivery apparatus 200. The actuation member 804 of the displacement control mechanism 800 extends from the handle 202 of the delivery apparatus 200, extends through the shaft 206, and is coupled to the coupling member 802 at its distal end portion. The proximal end portion of the actuation member 804 is coupled to the actuation mechanism 220 and the release mechanism 222 of the delivery apparatus 200, which are coupled to and/or disposed in the handle 202. The gear assembly 806 of the displacement control mechanism 800 is disposed within the distal end portion of the shaft 206. In other embodiments, the gear assembly 806 can be disposed adjacent the distal end portion of the shaft 206 rather than within the shaft 206.

In use, axial movement of the actuation member 804 relative to the shaft 206 moves the coupling member 802 and the actuation shafts 210 axially (e.g., to expand a prosthetic valve), and rotational movement of the actuation member 804 relative to the shaft 206 rotates the gear assembly 806 and the actuation shafts 210 (e.g., to release a prosthetic valve from the delivery apparatus 200). Additional details regarding the displacement control mechanism 800 and its components are provided below.

The coupling member 802 can comprise a plurality of openings (not shown) that extend axially therethrough (e.g., similar to the openings 708 of the coupling member 702). Referring to FIG. 33, the openings of the coupling member 802 are configured such that the actuation shafts 210 can extend through and rotate freely relative to the coupling member 802.

Distal end portions of the actuation shafts 210 are coupled to the coupling member 802 such that they cannot move axially relative to the coupling member 802. This can be accomplished by fixedly coupling peripheral gears 808 of the gear assembly 806 to the actuation shafts 210 either on the proximal side (as shown) or the distal side of the coupling member 802. The peripheral gears 808 are radially larger than the openings of the coupling member 802. As such, the peripheral gears 808 of the gear assembly 806 restrict relative axial movement between the actuation shafts 210 and the coupling member 802 in a first direction (e.g., distal in the illustrated configuration). To restrict relative axial movement in a second, opposite direction (e.g., proximal), stopper members (not shown, but see the stopper members 710 in FIGS. 25-26) can be coupled to the actuation shafts 210 on a side of the coupling member 802 opposite the peripheral gears 808. Accordingly, the actuation shafts 210 move axially together with the coupling member 802, the actuation member 804, the gear assembly 806, and the stopper members.

In the illustrated embodiment, the actuation shafts 210 extend from locations distal to the support sleeves 208, through the support sleeves 208, through the coupling member 802, through the peripheral gears 808, through the shaft 206, and to the handle 202. In such embodiments, the proximal end portions of the actuation shafts 210 can move axially relative to each other and relative to the handle 202. This allows the actuation shafts 210 to move axially relative to each other to accommodate the various path lengths of each actuation shaft (e.g., when the actuation shafts bend around a curve). Also, moving a single component (i.e., the actuation member 804) results in simultaneous movement of all of the actuation shafts (via the coupling member 802) along a constant (or at least substantially constant) distance, even when the positions of the proximal end portions of each actuation shaft 210 are different. As a result, the displacement control mechanism 800 can help to ensure uniform radial expansion of the prosthetic valve, even when the delivery apparatus is disposed in a curved configuration.

In other embodiments, the actuation shafts 210 can be relatively short. In such embodiments, the distal end portions of the actuation shafts 210 can extend beyond the distal ends of the support sleeves 208, and the proximal end portions of the actuation shafts 210 can be coupled to the peripheral gears 808 of the displacement control mechanism 800. Due to the relatively short length of the actuation shafts, the actuation shafts are less likely to be positioned around a curve in the patient's anatomy during expansion of the prosthetic valve. This reduces the need to allow the actuation shafts to move axially relative to each other, while still providing uniform expansion of the prosthetic valve.

The actuation member 804 is fixedly coupled to a central gear 810 of the gear assembly 806. Accordingly, the actuation member 804 and the central gear 810 move axially and rotate together. The central gear 810 is coupled to the coupling member 802 such that it can rotate relative to the coupling member 802 and such that it is restricted from moving axially relative to the coupling member 802. For example, in some embodiments, the central gear 810 can be mounted to the coupling member 802 via a bearing.

The actuation shafts 210 and the actuation member 804 can be coupled to the peripheral gears 808 and the central gear 810, respectively, in various manners. For example, this includes fasteners 812, adhesive, welding, and/or other means for coupling. In some embodiments, the actuation shafts 210, the actuation member 804, and/or the gears 808, 810 can comprise non-circular mating features (e.g., flats on the actuation shafts 210 and/or actuation member 804) to facilitate coupling and/or to prevent relative rotational movement therebetween.

In the illustrated embodiment, the gear assembly 806 is disposed on the proximal side of the coupling member 802. In other embodiments, the gear assembly 806 can be disposed on the distal side of the coupling member 802. In such embodiments, the coupling member 802 can comprise a central opening configured such that the actuation member 804 can extend therethrough and can rotate therein. The central gear 810 can prevent the actuation member 804 from moving proximally relative to the coupling member 802, and a stopper member can be disposed on the proximal side of the coupling member 802 to prevent the actuation member 804 from moving distally relative to the coupling member 802.

The peripheral gears 808 of the gear assembly 806 comprise teeth which mesh with teeth of the central gear 810 of the gear assembly 806. It should be noted that the peripheral gears 808 are restricted from rotating about the central axis of the central gear 810 (i.e., orbiting). Accordingly, rotation of the central gear 810 about its axis results in the rotation of the peripheral gears 808 about their respective axes. Rotating the central gear 810 in a first direction (e.g., clockwise) about its axis results in the peripheral gears 808 rotating in a second direction (e.g., counterclockwise) about their respective axes, and vice versa.

A prosthetic valve (e.g., the prosthetic valve 100) can be coupled to the delivery apparatus 200 having the displacement control mechanism 800, in a manner similar to that shown in FIG. 13. The prosthetic valve 100 can be compressed and loaded into the shaft 206 (see FIGS. 14-15), and the prosthetic valve 100 can be inserted into a patient's vasculature, advanced to or adjacent an implantation location, and deployed from the shaft 206 (see FIGS. 16-17). The prosthetic valve 100 can be expanded by moving the actuation member 804 of the displacement control mechanism 800 proximally relative to the shaft 206, which in turn moves the coupling member 802 and the actuation shafts 210 relative to the shaft 206 and moves the rack members 120 of the actuators 106 relative to the housing members 122 of the actuators to expand the frame 102 of the prosthetic valve 100. The actuation member 804 can be moved proximally by actuating the actuation mechanism 220 and/or by manually moving the actuation member 804 proximally relative to the handle 202. Once the prosthetic valve 100 is expanded and secured at the implantation location (e.g., in the native annulus), the prosthetic valve 100 can be released from the delivery apparatus 200 by rotating the actuation member 804 of the displacement control mechanism 800 about its axis relative to the shaft 206, which rotates the central gear 810 about its axis and which rotates the peripheral gears 808 and the actuation shafts 210 about their axes. This uncouples the actuation shafts 210 from the rack members 120 of the actuators 106. The actuation member 804 can be rotated by actuating the release mechanism 222 and/or by manually rotating the actuation member 804 relative to the handle 202.

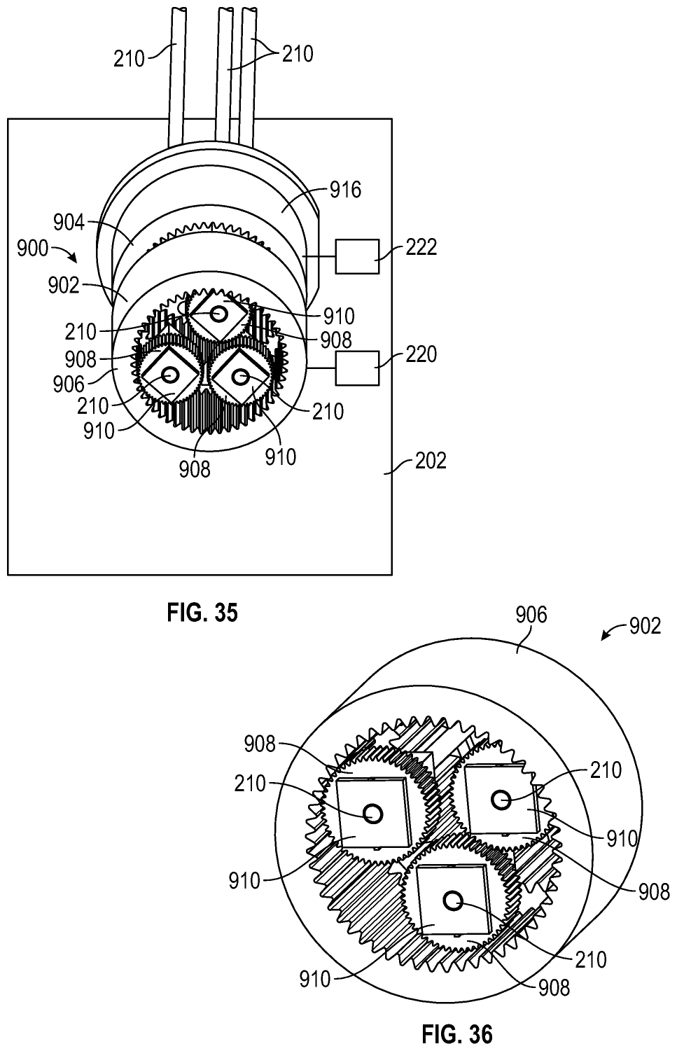

FIGS. 35-40 show a displacement control mechanism 900 and its components, according to another embodiment. Like the displacement control mechanisms 700 (and 800 and the force control mechanisms 400, 506, 606), the displacement control mechanism 900 allows the proximal end portions of the actuation shafts of the delivery apparatus to move axially relative to each other. The displacement control mechanism 900 therefore helps to ensure that the actuation shafts move the actuators of a prosthetic valve a constant distance and uniformly expand the prosthetic valve. The displacement control mechanism 900 allows the actuation shafts to be simultaneously moved axially, which can also help to ensure uniform expansion of the prosthetic valve. Additionally, the displacement control mechanism 900 allows the actuation shafts to be simultaneously rotated and thus released from the actuators of the prosthetic valve.

As shown in FIG. 35, the displacement control mechanism 900 can be coupled to and/or disposed within a handle of a delivery apparatus, such as the handle 202 of the delivery apparatus 200. The displacement control mechanism 900 comprises a first gear assembly 902 and a second gear assembly 904. The first gear assembly 902 is movably coupled to the actuation shafts 210 and is configured to translate rotational movement of the first gear assembly 902 into axial movement of the actuation shafts 210 (e.g., for expanding a prosthetic valve). As such, the first gear assembly 902 can also be referred to as “the expansion gear assembly.” The second gear assembly 904 is fixedly coupled to the actuation shafts 210 and is configured such that rotation of the second gear assembly 904 results in rotation of the actuation shafts 210 (e.g., for releasing a prosthetic valve from the delivery apparatus). The second gear assembly 904 can thus also be referred to as “the release gear assembly.”

Referring to FIG. 36, the first gear assembly 902 of the displacement control mechanism 900 comprises a first outer gear 906 and a plurality of first inner gears 908 disposed within and engaged with the first outer gear 906. As shown schematically in FIG. 35, the first outer gear 906 is coupled to the actuation mechanism 220 of the delivery apparatus 200. For example, in some embodiments, the first outer gear 906 can be coupled to an electric motor of the actuation mechanism 220 that is configured to rotate the first outer gear 906 about its axis and relative to the handle 202. In other embodiments, the first outer gear 906 can be coupled to or form an actuation knob of the actuation mechanism 220, which can be manually rotated relative to the handle 202.

Referring still to FIG. 36, the first outer gear 906 comprises an axial length that is longer than the axial length of the first inner gears 908. This allows the first inner gears 908 to remain engaged with the first outer gear 906 as the proximal end portions of the actuation shafts 210 move axially relative to the first outer gear 906 (e.g., when the delivery apparatus is curved and the actuation shafts travel different path lengths).

The first inner gears 908 can be coupled to respective actuation shafts 210 such that relative rotational movement between the first inner gears 908 and the actuation shafts 210 results in relative axial movement between the first inner gears 908 and the actuation shafts 210. For example, as shown in FIG. 39, the first gear assembly 902 comprises inserts 910 fixedly coupled to respective first inner gears 908. The inserts 910 comprise a threaded bore 912 configured to engage corresponding threads on the proximal end portion of the actuation shafts 210.

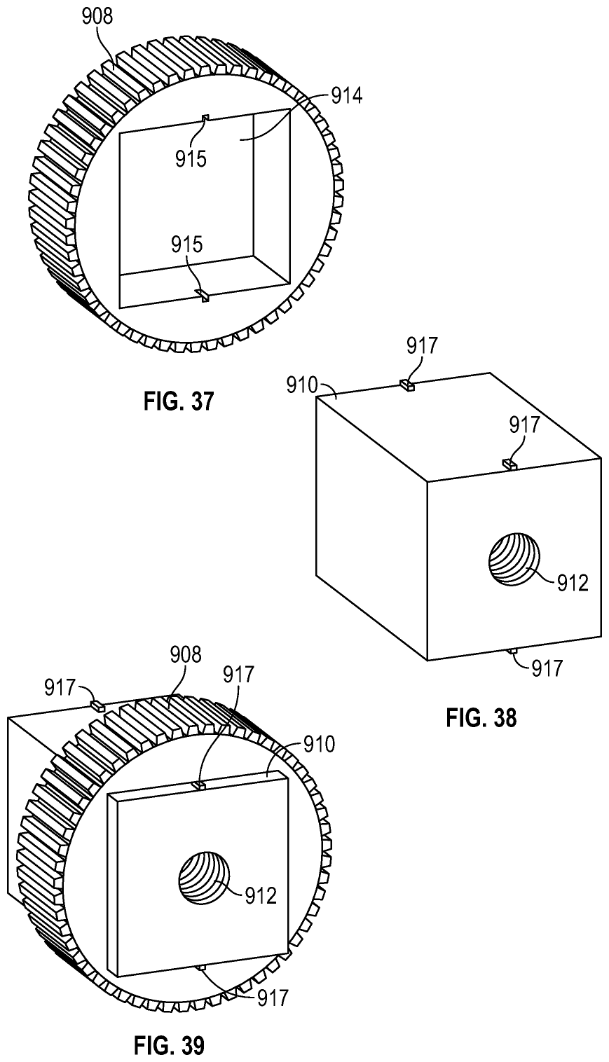

The first inner gears 908 and the inserts 910 can be coupled together in a manner configured to restrict relative rotational and/or axial movement therebetween. For example, the first inner gears 908 and the inserts 910 can be coupled together with adhesive, welding, mating features, and/or other means for coupling. For example, as shown in FIGS. 37-39, the first inner gears 908 and the inserts 910 comprise mating features configured to restrict relative rotational movement therebetween. Specifically, each of the first inner gears 908 comprises a non-circular (e.g., square) opening 914 corresponding to a non-circular (e.g., square) outer surface of the insert 910. Each of the first inner gears 908 also comprises slots 915 configured to receive corresponding tabs 917 of the insert 910. The non-circular shapes and/or the slots and tabs restrict relative rotational and/or axial movement between the first inner gears 908 and their respective inserts 910. In other instances, various other non-circular shapes (e.g., polygon, oval, etc.) and/or other types of mating features (e.g., a “slot and key” connection) can be used to restrict relative rotational and/or axial movement between the first inner gears 908 and their respective inserts 910.

Referring to FIGS. 35-36, rotating the first outer gear 906 about its central axis relative to the handle 202 results in rotation of the first inner gears 908 and the inserts 910 about their respective axes. The actuation shafts 210 do not rotate together with the inserts 910 because they are restricted from such motion by the second gear assembly 904. Thus, the actuation shafts 210 move axially relative to the inserts 910 as the gears 906, 908 and insert 910 rotate due to the threaded connection between the actuation shafts 210 and the inserts 910. When the distal end portions of the actuation shafts 210 are coupled to actuators of a prosthetic valve, axial movement of the actuation shafts 210 results in expansion/contraction of the prosthetic valve.

The threads of the proximal end portion of the actuation shafts 210 and the threaded bores 912 of the inserts 910 can be configured such that rotating the gears 906, 908 in a desired rotational direction (e.g., clockwise/counterclockwise) results in the actuation shafts 210 moving in a desired a desired axial direction (e.g., proximal/distal). For example, in some embodiments, the threads of the proximal end portion of the actuation shaft 210 and the threaded bore 912 of the insert 910 can be right-handed threads. In such embodiments, rotating the gears 906, 908 clockwise moves the actuation shafts proximally (e.g., to radially expand a prosthetic valve), and rotating the gears 906, 908 counterclockwise moves the actuation shafts distally (e.g., to radially contract a prosthetic valve). In other embodiments, the threads of the proximal end portion of the actuation shaft 210 and the threaded bore 912 of the insert 910 can be left-handed threads. In those embodiments, rotating the gears 906, 908 counterclockwise moves the actuation shafts proximally (e.g., to radially expand a prosthetic valve), and rotating the gears 906, 908 clockwise moves the actuation shafts distally (e.g., to radially contract a prosthetic valve).

In lieu of the inserts 910, the first inner gears 908 can comprise a threaded bore configured to directly engage corresponding threads on the proximal end portions of the actuation shafts 210. In yet other embodiments, the proximal end portions of the actuation shafts 210 can have threaded members (e.g., sleeves) fixedly coupled thereto (e.g., with adhesive, welding, fasteners, etc.). The threaded members can be configured to threadably engage respective threaded bores 912 of the inserts 910 or respective threaded bores of the first inner gears 908.

Various thread pitches or thread counts (“TPI”) can be used for the threads of the proximal end portion of the actuation shafts 210 and the threaded bores 912 of the inserts 910 to alter the axial distance the actuation shafts travel with each revolution of the inner gears 908. For example, smaller thread pitch/higher thread count produces less axial movement of the actuation shafts per revolution of the inner gears 908. Conversely, larger thread pitch/lower thread count produces more axial movement of the actuation shafts per revolution of the inner gears 908.

Various diameters and/or the gear ratio of the gears 906, 908 can also be used to alter the axial distance of the actuation shafts 210 travel with each revolution of the gears 906, 908.

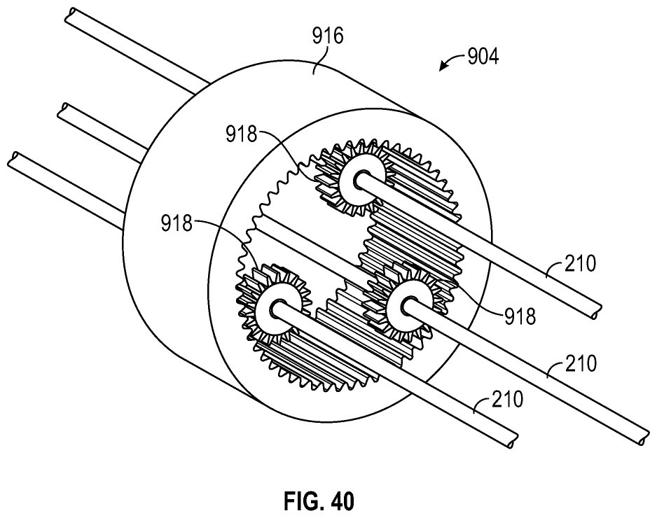

As shown in FIG. 40, the second gear assembly 904 of the displacement control mechanism 900 comprises a second outer gear 916 and a plurality of second inner gears 918 disposed within and engaged with the second outer gear 916. Generally speaking, the second gear assembly 904 of the displacement control mechanism 900 can be configured and function similar to the gear assembly 706 of the displacement control mechanism 700 in that it is configured to allow the proximal end portions of the actuation shafts 210 to move axially to accommodate differing path lengths traveled by the actuation shafts (e.g., due to curvature in the shaft 206) and to simultaneously rotate the actuation shafts upon rotation of the second outer gear 916 (e.g., to release a prosthetic valve from the delivery apparatus).

As shown schematically in FIG. 35, the second outer gear 916 can be coupled to and/or form a component of the release mechanism 222 of the delivery apparatus 200. For example, in some embodiments, the second outer gear 916 can be coupled to an electric motor of the release mechanism 222 that is configured to rotate the second outer gear 916 relative to the handle 202. In other embodiments, the second outer gear 916 can be coupled to or form a release knob of the release mechanism 222, which can be manually rotated relative to the handle 202.

Referring again to FIG. 40, the second outer gear 916 can comprise an axial length that is longer than the axial length of the second inner gears 918. This allows the second inner gears 918 to remain engaged with the second outer gear 916 as the proximal end portions of the actuation shafts 210 move axially relative to the second outer gear 916 (e.g., due to different path lengths traveled by the actuation shafts).

The second inner gears 918 can be fixedly coupled to respective actuation shafts 210 such that the second inner gears 918 and the actuation shafts 210 move together both axially and rotationally. The second inner gears 918 can be fixedly coupled to the actuation shafts 210 in various ways, including fasteners (e.g., a set screw and/or keyed connection), welding, adhesive, corresponding non-circular shapes, and/or other means for coupling.

The second gear assembly 904 can be used to release/couple the actuation shafts 210 from/to a prosthetic valve. For example, rotating the gears 916, 918 in a first direction (e.g., clockwise) rotates the actuation shafts 210 in the first direction and can result in the threads 242 on the distal end portions of the actuation shafts 210 engaging the threads of the rack members of the prosthetic valve (e.g., when the threads on the distal end portions of the actuation shafts and the rack member are right-handed threads). Rotating the gears 916, 918 in a second direction (e.g., counterclockwise) rotates the actuation shafts 210 in the second direction and can result in the threads 242 on the distal end portions of the actuation shafts 210 disengaging the threads of the rack members of the prosthetic valve (e.g., when the threads on the distal end portions of the actuation shafts and the rack member are right-handed threads).

During rotation of the first gear assembly 902 (e.g., when expanding/contracting a prosthetic valve), the second gear assembly 904 can be prevented from rotating together with the first gear assembly 902. This can be done either actively (e.g., with a locking mechanism) or passively (e.g., due to sufficient static friction in the second gear assembly 904). Accordingly, the second gear assembly 904 can help prevent the actuation shafts 210 from rotating together with the first inner gears 908 and inserts 910 of the first gear assembly 902, which in turn facilitates axial movement of the actuation shafts 210 relative to the inserts 910 due to the treaded connection between the actuation shafts 210 and the inserts 910. The second inner gears 918 can also move axially relative to the second outer gear 916 as the proximal end portions of the actuation shafts 210 move axially either together due to rotation of the first gear assembly 902 (e.g., during valve expansion/contraction) or individually in response to the actuation shafts traveling along paths of differing lengths (e.g., when disposed in the aortic arch).

In the illustrated embodiment, the first gear assembly 902 is disposed proximal to the second gear assembly 904. In other embodiments, the first gear assembly 902 can be disposed distal to the second gear assembly 904.

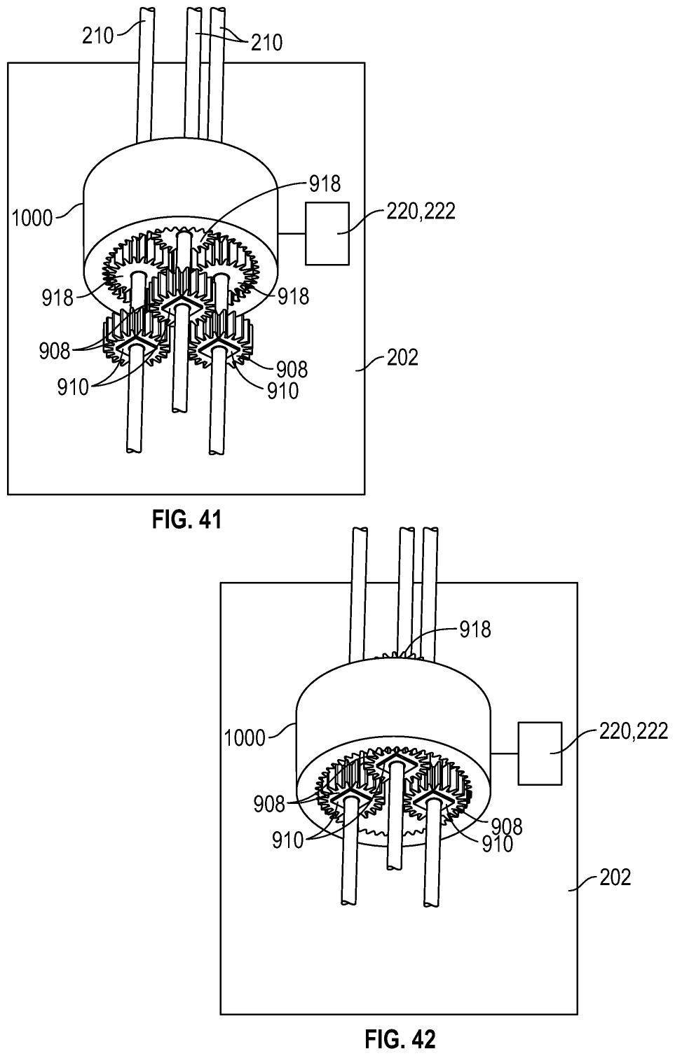

FIGS. 41-42 show a slidable outer gear 1000 that can be used, for example, with the displacement control mechanism 900 in lieu of the first and second outer gears 906, 916. The slidable outer gear 1000 can be moved axially (i.e., slid) between a first position and a second position. In the first position (FIG. 41), the slidable outer gear 1000 engages the first inner gears 908 and is disengaged from the second inner gears 918. Rotating the slidable outer gear 1000 (manually and/or via the actuation mechanism 220) while it is in the first position rotates the first inner gears 908 and moves the actuation shafts 210 axially relative to the first inner gears 908 (e.g., to expand or contract a prosthetic valve). The first position can thus also be referred to as “an expansion position” or “an expansion mode.” In the second position (FIG. 42), the slidable outer gear 1000 engages the second inner gears 918 and is disengaged from the first inner gears 908. Rotating the slidable outer gear 1000 while it is in the second position rotates the second inner gears 918 and also the actuation shafts 210 (e.g., for releasing/coupling a prosthetic valve). As such, the second position can also be referred to “a release position” or “a release mode.”

The slidable outer gear 1000 can provide several advantages. For example, it can reduce the number of components of the displacement control mechanism 900. It can also enhance safety by reducing the likelihood of a user inadvertently releasing a prosthetic valve from the delivery apparatus. For example, in some embodiments, the displacement control mechanism 900 can comprise a biasing member (e.g., a spring), a locking element (e.g., a switch and/or a groove), and/or other feature configured to position and/or retain the slidable outer gear 1000 in the expansion position (FIG. 41) by default. To release the prosthetic valve, the user would have to deliberately move the slidable outer gear 1000 to the release position (FIG. 42) by overcoming the bias, lock, etc., thereby reducing the likelihood of inadvertent release of the prosthetic valve.

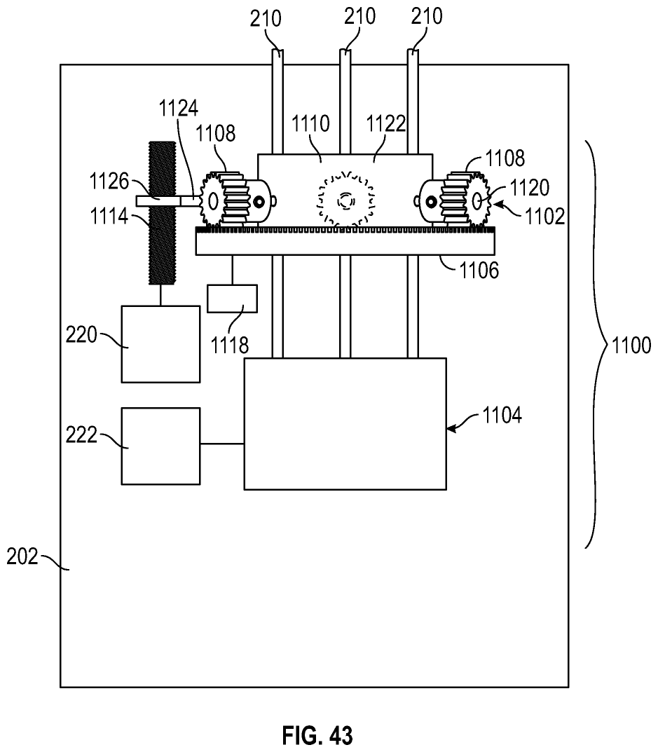

FIGS. 43-47 show a displacement control mechanism 1100, according to yet another embodiment. As shown in FIG. 43, the displacement control mechanism 1100 can be used, for example, with the delivery apparatus 200. The displacement control mechanism 1100 can be coupled to the proximal end portions of the actuation shafts 210 of the delivery apparatus 200 and disposed in the handle 202 of the delivery apparatus 200. In one mode of operation, the displacement control mechanism 1100 allows the proximal end portions of the actuation shafts 210 to move axially relative to the displacement control mechanism 1100 and relative to each other (e.g., when the actuation shafts travel different path lengths due to the actuation shafts being curved). In a second mode of operation, the displacement control mechanism 1100 can be used to simultaneously move the actuation shafts 210 axially relative to the shaft 206 and the support sleeves 208 (not shown) (e.g., for expanding/contracting a prosthetic valve). In a third mode of operation, the displacement control mechanism 1100 can be used to simultaneously rotate the actuation shafts 210 relative to the shaft 206 and the support sleeves 208 (e.g., for releasing/coupling a prosthetic valve).

Referring still to FIG. 43, the displacement control mechanism 1100 comprises a first gear assembly 1102 and a second gear assembly 1104. The first gear assembly 1102 can be coupled to and/or form a component of the actuation mechanism 220 of the delivery apparatus 200. The second gear assembly 1104 can be coupled to and/or form a component of the release mechanism 222 of the delivery apparatus 200.

In the illustrated embodiment, the first gear assembly 1102 is disposed distal to the second gear assembly 1104. In other embodiments, the first gear assembly 1102 can be disposed proximal to the second gear assembly 1104.

The first gear assembly 1102 can be moved between an unlocked configuration and a locked configuration. When the first gear assembly 1102 is in the unlocked configuration, the proximal end portions of the actuation shafts 210 can move freely (axially and/or rotationally) relative to the first gear assembly 1102 and move axially relative to the second gear assembly 1104 (e.g., to allow the actuation shafts to adjust to different relative path lengths and/or for releasing/coupling a prosthetic valve to the delivery apparatus). Also, when the first gear assembly 1102 is in the unlocked configuration, the second gear assembly 1104 can be used to simultaneously rotate the actuation shafts 210 relative to the shaft 206 and the support sleeves 208 (e.g., for releasing/coupling a prosthetic valve to the delivery apparatus). When the first gear assembly 1102 is in the locked configuration, the actuation shafts 210 are fixed (axially and rotationally) relative to the first gear assembly 1102 and relative to each other, and the first gear assembly 1102 can be used to simultaneously move the actuation shafts 210 axially relative to the second gear assembly 1104, the shaft 206, and the support sleeves 208 (e.g., for expanding/contracting a prosthetic valve). Additional details about the first and second gear assemblies 1102, 1104 and their operation are provided below.

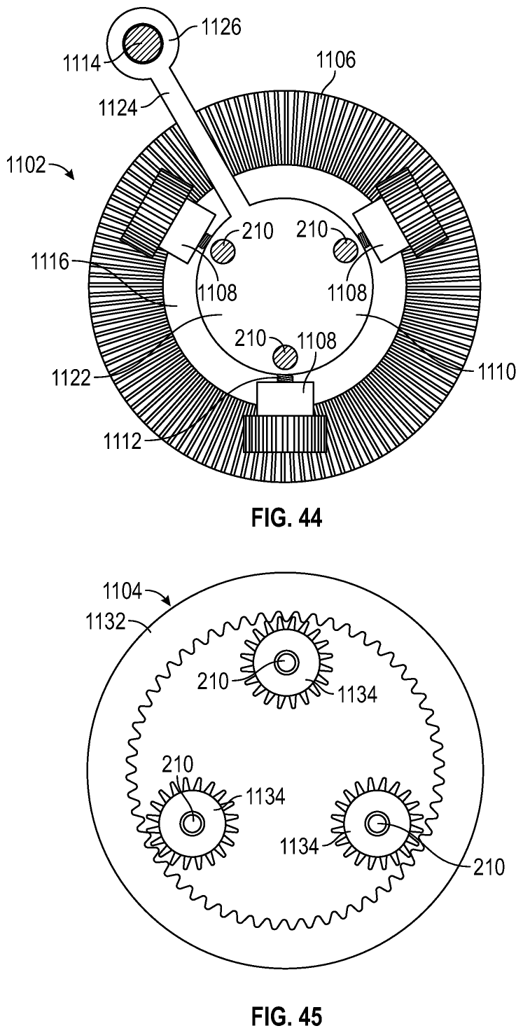

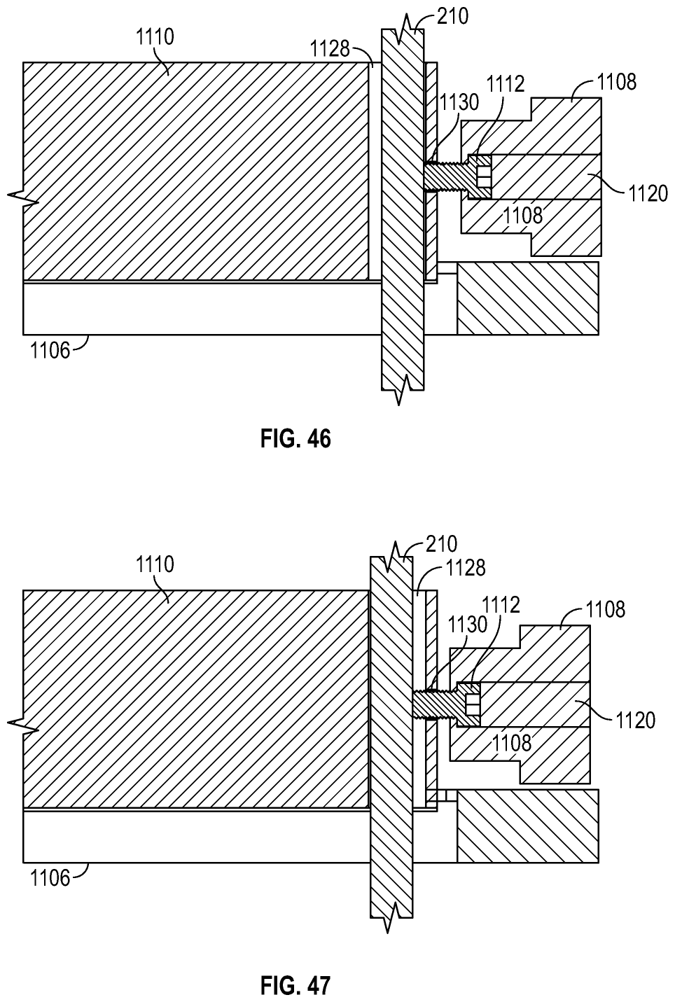

Referring to FIGS. 43-44, the first gear assembly 1102 comprises a face gear 1106, a plurality of first spur gears 1108 (e.g., three), a carriage member 1110, a plurality of locking screws 1112 (FIG. 46), and a drive screw 1114. The face gear 1106 and the spur gears 1108 comprise teeth configured to mesh together such that rotating the face gear 1106 about its axis causes the spurs gears 1108 to rotate about their respective axes. The carriage member 1110 is coupled to the spur gears 1108 by the locking screws 1112 (see FIG. 46). The carriage member 1110 can be selectively coupled to the actuation shafts 210 via the locking screws 1112 (see FIGS. 46-47). The carriage member 1110 can also be movably coupled to the drive screw 1114 such that rotation of the drive screw 1114 about its axis and relative to the carriage member 1110 results in axial movement of the carriage member 1110 (and axial movement of the actuation shafts 210 when they are coupled to the carriage member 1110).

The face gear 1106 of the first gear assembly 1102 can comprise teeth disposed on an axially-facing surface configured to engage corresponding teeth of the spur gears 1108. In some embodiments, the face gear 1106 and the spur gears 1108 can be beveled (also referred to as “bevel gears”). In the illustrated embodiment, the teeth of the face gear 1106 are disposed on the distal-facing surface of the face gear 1106. In other embodiments, the teeth of the face gear 1106 can be disposed on the proximal-facing surface of the face gear 1106.

Referring to FIG. 44, the face gear 1106 has an annular shape with a central opening 1116 in which the carriage member 1110 is disposed and through which the actuation shafts 210 can axially extend. The central opening 1116, among other things, allows the face gear 1106 to rotate about its axis relative to the carriage member 1110 and the actuation shafts 210. The face gear 1106 can be rotated manually and/or via a motor 1118 (FIG. 43).

As shown in FIG. 46, each of the spur gears 1108 comprises a central bore 1120 configured for receiving the locking screws 1112. The spur gear 1108 also comprises an annular shoulder extending radially inwardly into the central bore 1120. The shoulder is configured to allow the shaft portion of the locking screw 1112 to extend past the shoulder and into the carriage member 1110. The shoulder is also configured to engage the head portion of the locking screw 1112 such that the head portion of the locking screws 1112 cannot pass completely through the central bore 1120.

The locking screws 1112 are fixedly coupled to their respective spur gears 1108 such that locking screws 1112 move together (rotationally and axially) with their respective spur gears 1108. For example, in some embodiments, the central bores of the spur gears can comprise non-circular cross-sectional shapes (e.g., square, hexagonal, etc.), and the heads of the locking screws can comprise corresponding non-circular cross-sectional shapes. Additionally or alternatively, the locking screws can be fixedly coupled to their respective spur gears in various other ways including: fasteners (e.g., a set screw), adhesive, welding, etc. In yet other embodiments, a locking screw and a spur gear can be integrally formed as a unitary structure. For example, the locking screw can be a threaded shaft portion of the unitary structure extending from a spur gear portion of the unitary structure. In such embodiments, the central bore 1120 can be omitted.

Referring to FIGS. 43-44, the carriage member 1110 comprises a main body 1122, an extension arm 1124, and a connecting element 1126. The main body 1122 is radially aligned with the central opening 1116 of the face gear 1106. The extension arm 1124 extends radially outwardly from the main body 1122, and the connecting element 1126 extends radially outwardly from the extension arm 1124.

As shown in FIGS. 46-47, the main body 1122 of the carriage member 1110 comprises a plurality of axial openings 1128 and a plurality of radial openings 1130. The axial openings 1128 are configured for receiving the actuation shafts 210 and are configured such that the actuation shafts 210 can move freely relative to the main body 1122. The radial openings 1130 extend radially outwardly from the axial openings 1128 to an outer surface of the main body 1122. The radial openings 1130 are circumscribed by internal threads configured for engaging corresponding external threads of the locking screws 1112. Rotating the locking screws 1112 relative to the carriage member 1110 moves the locking screws 1112 into or out of the radial openings 1130 of the carriage member 1110 depending on the direction of rotation (e.g., clockwise/counterclockwise) and the configuration of the threads (e.g., right-handed/left-handed). This allows the locking screws 1112 to engage or disengage the actuation shafts 210, and thereby selectively restrict relative movement between the actuation shafts 210 and the carriage member 1110.

As shown in FIGS. 43-44, the connecting element 1126 of the carriage member 1110 comprises an aperture with internal threads configured to engage corresponding external threads of the drive screw 1114. Thus, rotation of the drive screw 1114 about its axis and relative to the connecting element 1126 results in axial movement of the carriage member 1110 along the drive screw 1114.

As mentioned above and referring again to FIGS. 46-47, the first gear assembly 1102 can be moved between the unlocked configuration (FIG. 46) and the locked configuration (FIG. 47) by moving the locking screws 1112 radially relative to the radial openings 1130 of carriage member 1110. The locking screws 1112 can be moved radially by rotating the spur gears 1108 about their respective axes and relative to the carriage member 1110. By virtue of the threaded connection, such rotation moves the locking screws 1112 relative to the carriage member 1110. The locking screws 1112 can be rotated relative to the carriage member 1110 by rotating the face gear 1106 about its axis and relative to the carriage member 1110, which in turn causes the spur gears 1108 and the locking screws 1112 to rotate together about their respective axes and relative to the carriage member 1110.

Rotating the face gear 1106 about its axis in a first direction (e.g., counterclockwise) relative to the carriage member 1110 results in the spur gears 1108 and the locking screws 1112 rotating about their respective axes in the first direction relative to the carriage member 1110. Counterclockwise rotation of the locking screws 1112 relative to the carriage member 1110 (when configured with right-handed threads) retracts the locking screws 1112 from the radial openings 1130 of the carriage member 1110. The locking screws 1112 can be retracted relative to the carriage member 1110 such that the locking screws 1112 do not obstruct the axial openings 1128 of the carriage member 1110, as shown in FIG. 46. This is the unlocked configuration of the first gear assembly 1102, which allows the actuation shafts 210 to move (axially and/or rotationally) freely relative to the carriage member 1110.

Rotating the face gear 1106 about its axis in a second direction (e.g., clockwise) relative to the carriage member 1110 results in the spur gears 1108 and the locking screws 1112 rotating about their respective axes in the second direction relative to the carriage member 1110. Clockwise rotation of the locking screws 1112 relative to the carriage member 1110 (when configured with right-handed threads) advances the locking screws 1112 into the radial openings 1130 of the carriage member 1110. The locking screws 1112 can be advanced relative to the carriage member 1110 such that the locking screws 1112 contact the actuation shafts 210 and urge the actuation shafts 210 radially inwardly against the inner walls of the carriage member 1110 that define the axial openings 1128, as shown in FIG. 47. This is the locked configuration of the gear assembly 1102, which restricts relative axial movement between the actuation shafts 210 and the carriage member 1110 due to the frictional engagement between the locking screws 1112, the actuation shafts 210, and the inner walls of the carriage member 1110.

The locking screws 1112 can be configured such that the actuation shafts 210 are not damaged when the locking screws 1112 contact the actuation shafts 210. For example, in some embodiments, the locking screws 1112 can comprise atraumatic tips configured to engage the actuation shafts 210 in a manner that does not result in damage to the actuation shafts 210.

Referring to FIG. 45, the second gear assembly 1104 can comprise an outer gear 1132 and a plurality of inner gears 1134 disposed radially within and engaging with the outer gear 1132. The second gear assembly 1104 can be configured and function similar to the second gear assembly 904 of the displacement control mechanism 900 and/or the gear assembly 706 of the displacement control mechanism 700. The outer gear 1132 of the second gear assembly 1104 comprises an axial length that is longer than the axial length of the inner gears 1134. This allows the inner gears 1134 to remain engaged with the outer gear 1132 as the proximal end portions of the actuation shafts 210 move axially relative to the outer gear 1132 (e.g., due to different path length of the actuation shafts and/or when expanding/compressing the prosthetic valve). The inner gears 1134 are fixedly coupled to respective actuation shafts 210 such that the inner gears 1134 and the actuation shafts 210 move together both axially and rotationally.

In this manner, the gear assembly 1104 can be used to release/couple the actuation shafts 210 from/to a prosthetic valve. For example, rotating the gears 1132, 1134 in a first direction (e.g., clockwise) rotates the actuation shafts 210 in the first direction and can result in the threads 242 on the distal end portions of the actuation shafts 210 engaging the threads of the rack members of the prosthetic valve (when the threads on the distal end portions of the actuation shafts and the rack member are right-handed threads) (see FIGS. 11-12). Rotating the gears 1132, 1134 in a second direction (e.g., counterclockwise) rotates the actuation shafts 210 in the second direction and can result in the threads 242 on the distal end portions of the actuation shafts 210 disengaging the threads of the rack members of the prosthetic valve (when the threads on the distal end portions of the actuation shafts and the rack member are right-handed threads).

The displacement control mechanism 1100 can be used, for example, with the delivery apparatus 200 and the prosthetic valve 100. With the prosthetic valve 100 coupled to the distal end portion of the delivery apparatus 200 and in a radially compressed configuration (see, e.g., FIGS. 13-15), the prosthetic valve can be inserted into a patient's vasculature (e.g., the patient's left femoral artery). The first gear assembly 1102 of the displacement control mechanism 1100 can be positioned in the unlocked position while the prosthetic valve 100 and the delivery apparatus 200 are advanced through the patient's vasculature to an implantation location (e.g., the patient's native aortic valve). The unlocked configuration of the first gear assembly 1102 allows the proximal end portions of the actuation shafts 210 to move axially relative to each other, the first gear assembly 1102, and the outer gear 1132 of the second gear assembly 1104 to adjust to the various path lengths the actuation shafts travel due to curvature in the shaft 206 of the delivery apparatus 200 (e.g., when the shaft 206 is disposed in the patient's aortic arch).

Once the prosthetic valve 100 is disposed at or adjacent to an implantation location, the first gear assembly 1102 of the displacement control mechanism 1100 can be moved from the unlocked configuration to the locked configuration by rotating the face gear 1106, the spur gears 1108, and the locking screws 1112 about their respective axes and relative to the carriage member 1110, as described above. With the first gear assembly 1102 in the locked configuration, the drive screw 1114 can be rotated about its axis in the first direction relative to the extension arm 1124 of the carriage member 1110, which moves the carriage member 1110 and the actuation shafts 210 proximally relative to the shaft 206 of the delivery apparatus 200. This results in radial expansion of the prosthetic valve 100. The prosthetic valve 100 can be recompressed (e.g., for repositioning and/or retrieval) by rotating the drive screw 1114 in the second, opposite direction. The drive screw 1114 can be rotated in the first and second directions in various ways, including by a motor or knob of the actuation mechanism 220.

When the prosthetic valve 100 is positioned and expanded with the patient as desired by the user, the prosthetic valve 100 can be locked in the radially expanded state and released from the delivery apparatus 200. This can be accomplished by moving the first gear assembly 1102 of the displacement control mechanism 1100 from the locked configuration to the unlocked configuration. This allows the actuation shafts 210 to move freely relative to the carriage member 1110. The outer gear 1132 of the second gear assembly 1104 can then be rotated about its axis relative to the handle 202, which results in the inner gears 1134 and the actuation shafts 210 rotating together about their respective axes. This results in the threads 242 at the distal end portion of the actuation shafts 210 retracting from the actuators 106 of the prosthetic valve 100. This releases the actuation shafts 210 from the prosthetic valve 100. The outer gear 1132 of the second gear assembly 1104 can be rotated relative to the handle 202 in various ways, including by a motor or knob of the release mechanism 222 and/or by rotating the outer gear 1132 directly. The delivery apparatus 200 can then be withdrawn from the patient's vasculature.

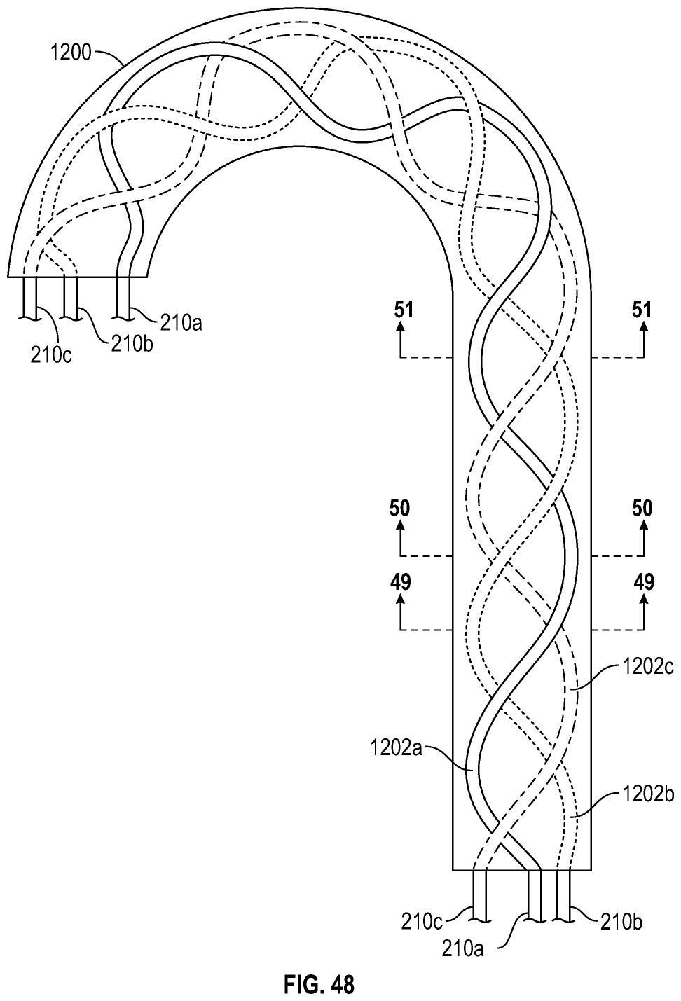

FIGS. 48-51 show a multi-lumen shaft 1200, according to one embodiment. The multi-lumen shaft 1200 (also referred to as “the shaft 1200”) can be used, for example, with the delivery apparatus 200 in lieu of the shaft 206. The shaft 1200 comprises a plurality of helical actuation lumens 1202a, 1202b, and 1202c (collectively and/or generically referred to as “the actuation lumens 1202”) and a central lumen 1204 disposed radially inwardly from the actuation lumens 1202. The actuation lumens 1202 can be configured to receive respective actuation shafts 210a, 210b, and 210c (collectively and/or generically referred to as “the actuation shafts 210”). The central lumen 1204 can be configured to receive the nosecone shaft 214. Although not shown, the shaft 1200 can comprise one or more other lumens, such as a recompression lumen.

Each of the actuation lumens 1202 extends from a proximal end of the shaft 1200 to a distal end of the shaft 1200 in a helical path. Configuring the shaft 1200 with the helical actuation lumens 1202 can, for example, help to ensure that each actuation shaft travels a similar axial path length even when the shaft 1200 is in a curved configuration (e.g., when the shaft 1200 is disposed within a patient's aortic arch). This can reduce stretching and/or help to ensure that stretching is at least substantially uniform in the actuation shafts 210 are curved. The actuation shafts 210 travel a similar distance because each actuation shaft 210 extending through the shaft 1200 is disposed at a first circumferential position of the shaft 1200 (e.g., a neutral position) for a first portion of its length, disposed at a second circumferential position (e.g., an outside position) of the shaft 1200 for a second portion of its length, and disposed at a third circumferential position (e.g., an inside position) of the shaft 1200 for a third portion of its length, as well as various circumferential positions between the first, second, and third circumferential positions. Accordingly, the distance each actuation shaft 210 travels through the shaft 1200 is the same as (or at least substantially similar to) the other actuation shafts 210 when the shaft 1200 is straight and when the shaft 1200 is curved. In this manner, the shaft 1200 can, for example, help to ensure that the prosthetic valve is evenly expanded.

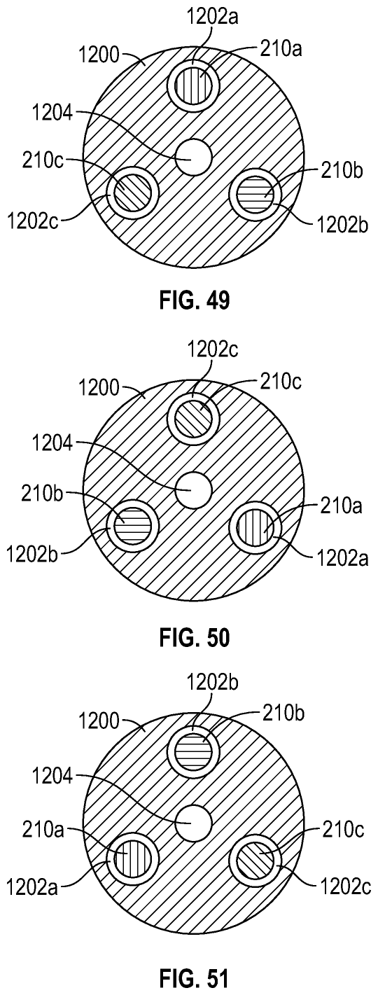

As used herein, the terms “neutral position and “neutral location” refer to a circumferential position of an actuation shaft when it is radially aligned with the plane of symmetry of a curved shaft through which the actuation shaft extends. For example, when the shaft 1200 is curved to the left (FIG. 48) or to the right, the neutral position for an actuation shaft is when it is at the 0/360-degree (12 o'clock) position (see, e.g., the position of the actuation shaft 210a in FIG. 49) and/or the 180-degree (6 o'clock) position. As used herein, the term “offset position/location” refers to any circumferential position of an actuation shaft when it is radially offset from the plane of symmetry of a curved shaft through which the actuation shaft extends. In other words, the offset position is any non-neutral position. As used herein, the term “outside position/location” refers to any circumferential position of an actuation shaft when it is radially offset to the outside of the plane of symmetry of a curved shaft through which the actuation shaft extends. For example, when the shaft 1200 is curved to the left (FIG. 48), an outside position for an actuation shaft is when it is at any position within the range of 1-179 degrees (with the 90-degree position being the outermost position—see, e.g., the position of the actuation shaft 210a in FIG. 50). As used herein, the term “inside position/location” refers to any circumferential position of an actuation shaft when it is radially offset to the inside of the plane of symmetry of a curved shaft through which the actuation shaft extends. For example, when the shaft 1200 is curved to the left (FIG. 48), an inside position for an actuation shaft is when it is at any position within the range of 181-359 degrees (with the 270-degree position being the innermost position—see, e.g., the position of the actuation shaft 210a in FIG. 51).

In some embodiments, all of the helical lumens 1202 can comprise the same pitch (i.e., the number of circumferential revolutions each actuation lumen makes per unit axial length of the shaft), and various pitches can be used. Providing a relatively high pitch for the actuation lumens 1202 can help to ensure that each of the actuation shafts 210 travels the same path length, even when the shaft 1200 is sharply curved. A high pitch can also increase the forces needed to move the actuation shafts axially (e.g., when expanding a prosthetic valve). As such, the pitch of the actuation lumens 1202 of the shaft 1200 can be selected to accommodate the extent to which the shaft 1200 will be curved during an implantation procedure, while also allowing the actuation shafts to be moved axially to expand the prosthetic valve. For example, in some embodiments, the pitch of the actuation lumens can be less than 200 mm. In some embodiments, the pitch of the actuation lumens can be less than 140 mm. In certain embodiments, the pitch of the actuation lumens can be 140 mm-70 mm. In particular embodiments, the pitch of the actuation lumens can be 125 mm-100 mm.

In the illustrated embodiment, the actuation lumens 1202 are evenly distributed relative to each other around the shaft 1200. In other words, there is about 120 degrees between adjacent actuation lumens 1202. In other embodiments, the actuation lumens 1202 can be non-evenly distributed relative to each other.

In some embodiments, a delivery apparatus can comprise the shaft and omit a force control mechanism and/or a displacement control mechanism. This is because the shaft 1200 helps to ensure the actuation shafts travel similar distances even when the shaft 1200 is curved. This can, for example, help to ensure that the prosthetic valve will be uniformly expanded when the actuation shafts are moved axially.

In other embodiments, the delivery apparatus 200 can comprise the shaft 1200, a force control mechanism, and/or a displacement control mechanism.

It should be noted that, although primarily shown and described in connection with the prosthetic valve 100 and the delivery apparatus 200, the force control mechanisms, the displacement control mechanisms, and the multi-lumen shafts disclosed herein can be used with various other prosthetic valves and/or delivery apparatus.

The disclosed delivery apparatus, components, and related methods for controlling the forces and/or displacement of the actuation shafts can, for example, help to ensure that the forces applied to the prosthetic heart valve by the delivery apparatus are evenly distributed. This can reduce the likelihood that the delivery apparatus and/or the prosthetic heart valve will become damaged during the implantation procedure. The disclosed delivery apparatus and methods can also help to ensure that the prosthetic heart valve is uniformly expanded. The delivery apparatus disclosed herein are also relatively simple and/or easy to use. This can, for example, reduce the risk of mistakes and/or reduce the time it takes to implant a prosthetic heart valve.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for implanting a prosthetic heart valve, the delivery apparatus comprising: a handle, a first shaft, a plurality of actuation shafts, and a control mechanism. The first shaft has a first end portion, a second end portion, and one or more lumens extending from the first end portion to the second end portion. The first end portion is coupled to the handle. The actuation shafts each have a proximal end portion and a distal end portion, and the actuation shafts extend through the one or more lumens of the first shaft. The control mechanism is coupled to the actuation shafts and to the handle. The control mechanism includes a first mode of operation and a second mode of operation. In the first mode of operation, the proximal end portions of the actuation shafts can move axially relative to each other and relative to the first shaft, and in the second mode of operation, the actuation shafts can be moved axially simultaneously.

Example 2. The delivery apparatus of any example herein, particularly example 1, wherein the control mechanism includes a force control mechanism.

Example 3. The delivery apparatus of any example herein, particularly example 2, wherein the force control mechanism comprises a pulley, wherein the proximal end portions of two of the actuation shafts are coupled together via the pulley, wherein the proximal end portions of the two of the actuation shafts move axially relative to each other and the pulley rotates when tension in the two of the actuation shafts is uneven.

Example 4. The delivery apparatus of any example herein, particularly example 2, wherein the plurality of actuation shafts includes a first actuation shaft, a second actuation shaft, and a third actuation shaft, wherein the force control mechanism comprises a carriage, a first pulley, a second pulley, and a third pulley, wherein the carriage is movable relative to the handle, wherein the first and second pulleys are rotatably mounted to the carriage, wherein the third pulley is fixed relative to the handle, wherein the proximal end portions of the first and second actuation shafts are coupled together via the first pulley, wherein the third actuation shaft extends around the second pulley and the third pulley, wherein the proximal end portions of the first and second actuation shafts move axially relative to each other and the first pulley rotates when tension in the first actuation shaft is different than tension in the second actuation shaft, and wherein the proximal end portion of the third actuation shaft moves relative to the first and second actuation shafts and the second and third pulleys rotate when tension in the third actuation shaft is different than tension in the first or second actuation shafts.

Example 5. The delivery apparatus of any example herein, particularly any one of examples 1-4, further comprising an actuation mechanism coupled to one of the actuation shafts and configured to move the actuation shafts axially simultaneously.

Example 6. The delivery apparatus of any example herein, particularly example 5, wherein the actuation mechanism comprises a rotatable knob, wherein rotation of the rotatable knob results in simultaneous axial movement of the actuation shafts.

Example 7. The delivery apparatus of any example herein, particularly example 5, wherein the actuation mechanism comprises an electric motor with a rotatable shaft, wherein rotation of the rotatable shaft results in simultaneous axial movement of the actuation shafts.

Example 8. The delivery apparatus of any example herein, particularly any one of examples 5-7, wherein the actuation mechanism comprises a spool configured for increasing and decreasing tension in the actuation shafts.

Example 9. The delivery apparatus of any example herein, particularly any one of examples 1-9, wherein the control mechanism includes a displacement control mechanism.

Example 10. The delivery apparatus of any example herein, particularly example 9, wherein the displacement control mechanism comprises a gear assembly having an outer gear and a plurality of inner gears, wherein the inner gears are coupled to respective actuation shafts, and wherein rotating the outer gear relative to the first shaft results in simultaneous rotational movement of the inner gears and the actuation shafts relative to the first shaft.

Example 11. The delivery apparatus of any example herein, particularly example 9, wherein the displacement control mechanism comprises a first gear assembly and a second gear assembly, wherein rotating the first gear assembly relative to the first shaft results in simultaneous axial movement of the actuation shafts relative to the first shaft, and wherein rotating the second gear assembly relative to the first shaft results in simultaneous rotational movement of the actuation shafts relative to the first shaft.

Example 12. The delivery apparatus of any example herein, particularly example 11, wherein the first gear assembly is coupled to an actuation mechanism, and wherein the second gear assembly is coupled to a release mechanism.

Example 13. The delivery apparatus of any example herein, particularly any one of examples 11-12, wherein the displacement control mechanism comprises a slidable outer gear configured to be moved between a first position and a second position, wherein in the first position, the slidable outer gear engages a plurality of first inner gears of the first gear assembly, and wherein in the second position, the slidable outer gear engages a plurality of second inner gears of the second gear assembly.

Example 14. The delivery apparatus of any example herein, particularly example 9, wherein the displacement control mechanism comprises a coupling member, an actuation member, and a gear assembly, wherein the coupling member is coupled to the distal end portions of the actuation shafts, wherein the actuation member extends through the first shaft, wherein a first end portion of the actuation member is coupled to the coupling member, and wherein the gear assembly is coupled to the proximal end portions of the actuation shafts, wherein axial movement of the actuation member relative to the first shaft results in simultaneous axial movement of the coupling member and the actuation shafts relative to the first shaft and the gear assembly, and wherein rotating the gear assembly relative to the first shaft results in simultaneous rotational movement of the actuation shafts relative to the first shaft.

Example 15. The delivery apparatus of any example herein, particularly example 14, wherein the actuation member is coupled to an actuation mechanism.

Example 16. A delivery assembly comprising the delivery apparatus of any example herein, particularly the delivery apparatus of any one of examples 1-15, and a mechanically-expandable prosthetic heart valve.

Example 17. The delivery assembly of any example herein, particularly example 16, wherein the mechanically-expandable prosthetic heart valve comprises a frame with a plurality of struts, and a plurality of actuators, wherein the struts of the frame are pivotably coupled together, and wherein the actuators are coupled to the struts of the frame and configured to move the frame between a radially compressed configuration and a radially expanded configuration.

Example 18. The delivery assembly of any example herein, particularly example 17, wherein the actuation shafts of the delivery apparatus are releasably coupled to the actuators of the prosthetic heart valve such that relative axial movement between the actuation shafts and the first shaft moves the frame of the prosthetic heart valve between the radially compressed configuration and the radially expanded configuration.

Example 19. A delivery apparatus comprising a handle, a first shaft, a plurality of actuation shafts, and a force control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The force control mechanism is coupled to the actuation shafts and to the handle. The force control mechanism is configured such that the proximal end portions of the actuation shafts can move axially relative to each other when the first shaft is curved.

Example 20. The delivery apparatus of any example herein, particularly example 19, wherein the force control mechanism comprises a pulley system interconnecting the actuation shafts.

Example 21. The delivery apparatus of any example herein, particularly example 20, wherein the pulley system includes one or more pulleys that are axially movable relative to the handle, and one or more pulleys that are axially fixed relative to the handle.

Example 22. A delivery apparatus comprising a handle, a first shaft, a plurality of actuation shafts, and a displacement control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The displacement control mechanism is coupled to the actuation shafts and to the handle. The displacement control mechanism is configured such that the proximal end portions of the actuation shafts can move axially relative to each other when the first shaft is curved.

Example 23. The delivery apparatus of any example herein, particularly example 22, wherein the displacement control mechanism comprises a gear assembly having an outer gear and a plurality of inner gears, wherein the inner gears are fixedly coupled to respective actuation shafts, and wherein rotating the outer gear relative to the first shaft results in simultaneous rotational movement of the inner gears and the actuation shafts relative to the first shaft.

Example 24. The delivery apparatus of any example herein, particularly example 23, wherein the outer gear comprises radially inwardly facing teeth having a first axial length, wherein the inner gears comprise radially outwardly facing teeth having a second axial length, and wherein the first axial length is greater than the second axial length such that the teeth of the inner gears remain engaged with the teeth of the outer gear when the actuation shafts move axially relative to each other.

Example 25. The delivery apparatus of any example herein, particularly example 24, wherein a ratio of the first axial length to the second axial length is within a range of 1.5-10.

Example 26. The delivery apparatus of any example herein, particularly example 24, wherein a ratio of the first axial length to the second axial length is within a range of 2-6.

Example 27. The delivery apparatus of any example herein, particularly example 24, wherein a ratio of the first axial length to the second axial length is within a range of 3-5.

Example 28. The delivery apparatus of any example herein, particularly example 24, wherein a ratio of the first axial length to the second axial length is within a range of 4-4.5.

Example 29. The delivery apparatus of any example herein, particularly example 29, wherein the displacement control mechanism comprises a gear assembly having an inner gear engaged with a plurality of peripheral gears disposed radially outwardly from the inner gear, wherein the gear assembly is spaced apart from the handle and disposed in or adjacent the distal end portion of the first shaft, wherein rotating the peripheral gear relative to the first shaft causes the peripheral gears to rotate relative to the first shaft, and wherein the peripheral gears are fixedly coupled to respective actuation shafts.

Example 30. The delivery apparatus of any example herein, particularly example 29, wherein the displacement control mechanism further comprises a coupling member and an actuation member, wherein the peripheral gears are rotatably coupled to the coupling member, wherein a first end portion of the actuation member is coupled to the coupling member, wherein a second end portion of the actuation member is disposed in the handle, and wherein axial movement of the actuation member relative to the first shaft results in simultaneous axial movement of the coupling member and the actuation shafts relative to the first shaft, and wherein rotating the actuation member relative to the first shaft results in simultaneous rotational movement of the inner gear, the peripheral gears, and the actuation shafts relative to the first shaft.

Example 31. The delivery apparatus of any example herein, particularly example 30, wherein the actuation member is coupled to an actuation mechanism.

Example 32. A delivery apparatus comprising a handle, a first shaft, and a plurality of actuation shafts. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft.

Example 33. A delivery apparatus comprises a handle, a first shaft, a plurality of actuation shafts, a force control mechanism, and a displacement control mechanism. The first shaft has a first end portion, a second end portion, and one or more lumens extending from the first end portion to the second end portion, and the first end portion is coupled to the handle. Each actuation shaft has a proximal end portion and a distal end portion, and the actuation shafts extend through the one or more lumens of the first shaft. The force control mechanism is coupled to the actuation shafts and to the handle. The force control mechanism is configured such that the proximal end portions of the actuation shafts can move axially relative to each other when the first shaft is curved. The displacement control mechanism is coupled to the actuation shafts and to the handle. The displacement control mechanism is configured such that the proximal end portions of the actuation shafts can move axially relative to each other when the first shaft is curved.

Example 34. A delivery apparatus comprises a handle, a first shaft, a plurality of actuation shafts, and a force control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The force control mechanism is coupled to the actuation shafts and configured to evenly distribute forces applied to the actuation shafts.

Example 35. A delivery apparatus comprises a handle, a first shaft, a plurality of actuation shafts, and a displacement control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The displacement control mechanism is coupled to the actuation shafts and configured such that the proximal end portions of the actuation shafts can move axially relative to each other when the first shaft is curved.

Example 36. A delivery apparatus comprising a handle, a first shaft, a plurality of actuation shafts, a force control mechanism, and a displacement control mechanism. The first shaft has a first end portion, a second end portion, and a plurality of helical lumens extending from the first end portion to the second end portion, and the first end portion is coupled to the handle. Each actuation shaft has a proximal end portion and a distal end portion, and the actuation shafts extend through respective helical lumens of the first shaft. The force control mechanism is coupled to the actuation shafts and configured to evenly distribute forces applied to the actuation shafts. The displacement control mechanism is coupled to the actuation shafts and configured such that the proximal end portions of the actuation shafts can move axially relative to each other when the first shaft is curved.

Example 37. A force control mechanism for a delivery apparatus for implanting a prosthetic heart valve is provided. The force control mechanism comprises a pulley system and a movable carriage. The pulley system is configured for interconnecting a plurality of actuation shafts of a delivery apparatus. The movable carriage is connected to the pulley system and is configured to be movably coupled to a handle of a delivery apparatus. The pulley system and the movable carriage are configured to move axially and/or rotationally to balance forces applied to and/or carried by the actuation shafts of the delivery apparatus.

Example 38. A force control mechanism for a delivery apparatus for implanting a prosthetic heart valve is provided. The force control mechanism comprises a first pulley, a second pulley, a third pulley, and a carriage. The first pulley is configured to be coupled to first and second actuation shafts of a delivery apparatus. The second pulley is configured to be coupled to a third actuation shaft of the delivery apparatus. The third pulley is configured to be coupled to the third actuation shaft of the delivery apparatus. The carriage is configured to be movably coupled to a handle of the delivery apparatus. The first and second pulleys are rotatably coupled to the carriage, and the carriage is axially movable relative to the third pulley. Proximal end portions of the first and second actuation shafts move axially relative to each other and the first pulley rotates when tension in the first and second actuation shafts is uneven. A proximal end portion of the third actuation shaft moves axially relative to the first and second actuation shafts and the second and third pulleys rotate when tension in the third actuation shaft and the first or second actuation shafts is uneven.

Example 39. A displacement control mechanism for a delivery apparatus configured for implanting a prosthetic heart valve is provided. The displacement control mechanism comprises one or more gear assemblies. The gear assemblies are configured to be coupled to actuation shafts of a delivery apparatus. The gear assemblies are configured to allow proximal end portions of the actuation shafts to move independently relative to each other in an axial direction, and configured to rotate the actuation shafts simultaneously about their respective axes.

Example 40. The displacement control mechanism of any example herein, particularly example 39, wherein the one or more gear assemblies comprise a first gear assembly configured to be disposed within or adjacent a distal end portion of a shaft of the delivery apparatus.

Example 41. The displacement control mechanism of any example herein, particularly example 40, wherein the first gear assembly comprises an inner gear and a plurality of peripheral gears circumscribing the inner gear.

Example 42. The displacement control mechanism of any example herein, particularly example 39, wherein the one or more gear assemblies comprise a first gear assembly configured to be disposed within a handle at a proximal end portion of the delivery apparatus.

Example 43. The displacement control mechanism of any example herein, particularly example 42, wherein the first gear assembly comprises a plurality of inner gears and an outer gear circumscribing the inner gears.

Example 44. The displacement control mechanism of any example herein, particularly any one of examples 42-43, wherein the one or more gear assemblies comprise a second gear assembly configured to be disposed within a handle at a proximal end portion of the delivery apparatus.

Example 45. The displacement control mechanism of any example herein, particularly example 44, wherein the second gear assembly comprises a plurality of inner gears and an outer gear circumscribing the inner gears.

Example 46. The displacement control mechanism of any example herein, particularly example 42, wherein the first gear assembly comprises a face gear and a plurality of spur gears.

Example 47. A shaft for a delivery apparatus configured for implanting a prosthetic heart valve is provided. The shaft comprises a plurality of helical lumens extending from a first end portion of the shaft to a second end portion of the shaft, and each helical lumen is configured to receive an actuation shaft of a delivery apparatus.

Example 48. The shaft of any example herein, particularly example 47, wherein each helical lumen is circumferentially spaced from an adjacent helical lumen.

Example 49. The shaft of any example herein, particularly any one of examples 47-48, wherein the shaft includes 3-15 helical lumens.

Example 50. The shaft of any example herein, particularly any one of examples 47-49, wherein the shaft includes 3-6 helical lumens.

Example 51. The shaft of any example herein, particularly any one of examples 47-50, wherein the shaft includes exactly three helical lumens.

The features described herein with regard to any example can be combined with other features described in any one or more of the other examples, unless otherwise stated. For example, any one or more of the features of the force control mechanism 400 can be combined with any one or more features of the force control mechanism 606. As another example, any one or more features of the displacement control mechanism 700 can be combined with any one or more features of the displacement control mechanism 900.

In view of the many possible embodiments to which the principles of the disclosure may be applied, it should be recognized that the illustrated embodiments are only examples and should not be taken as limiting the scope of the claims. Rather, the scope of the claimed subject matter is defined by the following claims and their equivalents.

We claim:

1. A delivery apparatus for a prosthetic heart valve, the delivery apparatus comprising:
a handle;
a first shaft comprising a first end portion, a second end portion, and one or more lumens, the one or more lumens extending from the first end portion to the second end portion, the first end portion coupled to the handle;
a plurality of actuation shafts, each actuation shaft of the plurality of actuation shafts comprising a proximal end portion and a distal end portion, the plurality of actuation shafts extending through the one or more lumens of the first shaft; and
a control mechanism coupled to the plurality of actuation shafts and to the handle,
wherein the control mechanism comprises a first mode of operation and a second mode of operation;
wherein, when in the first mode of operation, the proximal end portions of the plurality of actuation shafts are configured to move axially relative to each other and relative to the first shaft; and
wherein, when in the second mode of operation, the plurality of actuation shafts are configured to move axially simultaneously.

2. The delivery apparatus of claim 1, wherein the control mechanism includes a force control mechanism.

3. The delivery apparatus of claim 2, wherein the force control mechanism comprises a pulley, wherein the proximal end portions of two of the actuation shafts are coupled together via the pulley, wherein the proximal end portions of the two of the actuation shafts move axially relative to each other and the pulley rotates when tension in the two of the actuation shafts is uneven.

4. The delivery apparatus of claim 2, wherein the plurality of actuation shafts includes a first actuation shaft, a second actuation shaft, and a third actuation shaft, wherein the force control mechanism comprises a carriage, a first pulley, a second pulley, and a third pulley, wherein the carriage is movable relative to the handle, wherein the first and second pulleys are rotatably mounted to the carriage, wherein the third pulley is fixed relative to the handle, wherein the proximal end portions of the first and second actuation shafts are coupled together via the first pulley, wherein the third actuation shaft extends around the second pulley and the third pulley, wherein the proximal end portions of the first and second actuation shafts move axially relative to each other and the first pulley rotates when tension in the first actuation shaft is different than tension in the second actuation shaft, and wherein the proximal end portion of the third actuation shaft moves relative to the first and second actuation shafts and the second and third pulleys rotate when tension in the third actuation shaft is different than tension in the first or second actuation shafts.

5. The delivery apparatus of claim 1, further comprising an actuation mechanism coupled to one of the actuation shafts and configured to move the actuation shafts axially simultaneously.

6. The delivery apparatus of claim 5, wherein the actuation mechanism comprises a rotatable knob, wherein rotation of the rotatable knob results in simultaneous axial movement of the actuation shafts.

7. The delivery apparatus of claim 5, wherein the actuation mechanism comprises an electric motor with a rotatable shaft, wherein rotation of the rotatable shaft results in simultaneous axial movement of the actuation shafts.

8. The delivery apparatus of claim 5, wherein the actuation mechanism comprises a spool configured for increasing and decreasing tension in the actuation shafts.

9. The delivery apparatus of claim 1, wherein the control mechanism includes a displacement control mechanism.

10. The delivery apparatus of claim 9, wherein the displacement control mechanism comprises a gear assembly having an outer gear and a plurality of inner gears, wherein the inner gears are coupled to respective actuation shafts, and wherein rotating the outer gear relative to the first shaft results in simultaneous rotational movement of the inner gears and the actuation shafts relative to the first shaft.

11. The delivery apparatus of claim 9, wherein the displacement control mechanism comprises a first gear assembly and a second gear assembly, wherein rotating the first gear assembly relative to the first shaft results in simultaneous axial movement of the actuation shafts relative to the first shaft, and wherein rotating the second gear assembly relative to the first shaft results in simultaneous rotational movement of the actuation shafts relative to the first shaft.

12. The delivery apparatus of claim 11, wherein the first gear assembly is coupled to an actuation mechanism, and wherein the second gear assembly is coupled to a release mechanism.

13. The delivery apparatus of claim 11, wherein the displacement control mechanism comprises a slidable outer gear configured to be moved between a first position and a second position, wherein in the first position, the slidable outer gear engages a plurality of first inner gears of the first gear assembly, and wherein in the second position, the slidable outer gear engages a plurality of second inner gears of the second gear assembly.

14. The delivery apparatus of claim 9, wherein the displacement control mechanism comprises a coupling member, an actuation member, and a gear assembly, wherein the coupling member is coupled to the distal end portions of the actuation shafts, wherein the actuation member extends through the first shaft, wherein a first end portion of the actuation member is coupled to the coupling member, and wherein the gear assembly is coupled to the proximal end portions of the actuation shafts, wherein axial movement of the actuation member relative to the first shaft results in simultaneous axial movement of the coupling member and the actuation shafts relative to the first shaft and the gear assembly, and wherein rotating the gear assembly relative to the first shaft results in simultaneous rotational movement of the actuation shafts relative to the first shaft.

15. The delivery apparatus of claim 14, wherein the actuation member is coupled to an actuation mechanism.

16. A delivery assembly comprising:
a delivery apparatus comprising:
a handle;
a first shaft coupled to the handle and comprising one or more lumens;
a plurality of actuation shafts extending through the one or more lumens of the first shaft, each actuation shaft of the plurality of actuation shafts comprising a proximal end portion and a distal end portion; and
a control mechanism coupled to the plurality of actuation shafts and to the handle, the control mechanism comprising a first mode of operation and a second mode of operation; and
a mechanically-expandable prosthetic heart valve comprising a frame with a plurality of struts and a plurality of actuators,
wherein:
when the control mechanism is in the first mode of operation, the proximal end portions of the plurality of actuation shafts are configured to move axially relative to each other and relative to the first shaft; and
when the control mechanism is in the second mode of operation, the plurality of actuation shafts are configured to move axially simultaneously.

17. The delivery assembly of claim 16, wherein the struts of the frame are pivotably coupled together, and wherein the plurality of actuators are coupled to the struts of the frame and configured to move the frame between a radially compressed configuration and a radially expanded configuration.

18. The delivery assembly of claim 16, wherein the plurality of actuation shafts of the delivery apparatus are releasably coupled to the plurality of actuators of the mechanically-expandable prosthetic heart valve such that relative axial movement between the actuation shafts and the first shaft moves the frame of the mechanically-expandable prosthetic heart valve between the radially compressed configuration and the radially expanded configuration.

19. The delivery assembly of claim 16, wherein rotating the plurality of actuation shafts in a first direction is configured to couple the distal end portions of the plurality of actuation shafts with the plurality of actuators of the mechanically-expandable prosthetic heart, and wherein rotating the plurality of actuation shafts in a second direction is configured to de-couple the distal end portions of the plurality of actuation shafts from the plurality of actuators of the mechanically-expandable prosthetic heart.

20. The delivery assembly of claim 16, wherein the control mechanism comprises a gear assembly disposed at a distal end of the first shaft opposite of the handle, wherein the gear assembly comprises a central gear and a plurality of peripheral gears, wherein the plurality of peripheral gears are configured to rotate the plurality of actuators of the mechanically-expandable prosthetic heart valve.
